(19) United States
(12) Patent Application Publication
(10) Pub. No.: US 2003/0207840 A1
(43) Pub. Date: Nov. 6, 2003

(54) Genes induced by hypoxia

(75) Inventors: Gregory J. Riggins, Durham, NC (US); Anita Lal, Durham, NC (US)

Correspondence Address: BANNER & WITCOFF, 1001 G STREET N W, SUITE 1100, WASHINGTON, DC, 20001, US

(73) Assignee: Duke University, Durham, NC

(21) Appl. No.: 10/465,572

(22) Filed: Jun. 20, 2003

Related U.S. Application Data
(62) Division of application No. 10/201,642, filed on Jul. 24, 2002.
(60) Provisional application No. 60/307,600, filed on Jul. 26, 2001.

(51) Int. Cl. A61K 51/00; A61K 48/00; A61K 39/395

(52) U.S. Cl. 514/44; 424/1.49; 424/178.1; 424/155.1

(57) ABSTRACT

Solid tumors and other conditions related to angiogenesis, including wounds, bone fractures, follicular development, ischemia, retinopathy, psoriasis, and rheumatoid arthritis are treated or detected with reagents which either detect, promote, or disrupt expression of one or more of HOG18, HOG3, HOG8, PLOD2, CA9, HXB, IGFBP5, STC1, HFARP, mig-6, and SSR4. Each of these genes was found to be induced by hypoxia.

This application claims the benefit of provisional application serial No. 60/307,600 filed Jul. 26, 2001, the content of which is expressly incorporated herein.

The U.S. Government retains certain rights to this invention due to funding by the National Institutes of Health of NCI CGAP contract #S98-146.

### FIELD OF THE INVENTION

The invention is related to methods for the enhancement or inhibition of the expression of genes related to responses to hypoxia. More specifically, the invention is related to methods to increase or decrease the expression of certain genes which promote angiogenesis, the growth of tumors, wound healing, the growth and development of tissues such as bone or ovarian follicles, and inflammatory conditions such as arthritis or psoriasis.

### BACKGROUND OF THE INVENTION

Cellular responses to hypoxia have important effects on the development and metastasis of tumors, angiogenesis, wound healing, recovery from ischemia, and other physiological and pathological processes. Reduced oxygen availability can trigger a variety of cellular mechanisms including angiogenesis, cell-cycle arrest, apoptosis, and glycolysis.

The molecular mechanisms by which cells adapt to hypoxia are poorly understood. An initial response to hypoxia is increased levels of hypoxia-inducible factor 1 (HIF-1) protein (Semenza G L, J Appl Physiol 88:1474-80 (2000)). This transcription factor is a key regulator of hypoxia-driven apoptosis, growth arrest, and tumor vascularization. HIF-1 is additionally linked to oncogenesis by the Von Hippel-Lindau tumor suppressor protein (vHL), which controls HIF-1 levels by proteolysis (Maxwell P H, et al., Nature 399:271-5 (1999)). Vascular endothelial growth factor (VEGF) is a powerful hypoxia-induced mitogen for endothelial cell growth, which plays a critical role in the development of tumor vessels (Yancopoulos G D, et al., Nature 407:242-8 (2000)). Expression of the angiopoietin family of secreted proteins is also regulated by hypoxia (Krikun G, et al. Biochem Biophys Res Commun 275:159-63 (2000)). During angiogenesis, the angiopoietins function with VEGF and Tie2, an endothelial-specific receptor with tyrosine kinase activity. Angiopoietin-1 (ANG1) is involved in recruitment of peri-endothelial cells by emerging blood vessels and in the maintenance of cell-cell and cell matrix association in mature capillaries. Angiopoietin-2 (ANG2) behaves as an antagonist to ANG1, thus blocking the Tie2 signal. The combination of ANG2 and VEGF causes disruption of cell-cell association, which promotes the differentiation phase of angiogenesis (Audero E, et al. Arterioscler Thromb Vasc Biol 21:536-41(2001); Yancopoulos G D, et al., Nature 407:242-8 (2000)).

Inhibition of angiogenesis is thought to provide an opportunity for therapy of cancer and other conditions involving responses to hypoxia. Normal tissues maintain a balance between cellular proliferation and oxygen supply. This balance is altered in solid tumors, resulting in focal regions with reduced oxygen levels compared to surrounding normal tissue (Thrall D E, et al., Radiother Oncol 44:171-6 (1997)). The cells in hypoxic regions either adapt to the hypoxic stress or die. Adaptation to a low oxygen environment can have serious consequences. For example, hypoxic tumor cells have a higher resistance to radiotherapy and certain chemotherapies (Brown J M, Cancer Res 59:5863-70 (1999)). Hypoxia can promote a higher mutation rate (Yuan J, et al., Cancer Res 60:4372-6 (2000)) and select for a more metastatic and malignant phenotype (Hockel M, et al., Cancer Res 56:4509-15 (1996); Rofstad E K, Int J Radiat Biol 2000;76:589-605 (2000)). Tumor angiogenesis may be blocked by disrupting the expression of VEGF or its receptor (Schlaeppi J M, & Wood J M, Cancer Metastasis Rev 18:473-81 (1999)).

Angiogenesis can be either beneficial or problematic, depending upon the circumstances. In processes such as wound healing, bone healing, recovery from ischemia, and follicular development, angiogenesis provides beneficial increased vascularization. However, angiogenesis is problematic in disease states like retinopathy and conditions caused by inflammation such as rheumatoid arthritis and psoriasis. The ability to promote or inhibit angiogenesis provides a method for treating these disease states.

Thus, there is a need in the art for knowledge of genes whose expression is induced by hypoxia because the products of such genes modulate angiogenesis, tumor growth, and a variety of pathological conditions.

### SUMMARY OF THE INVENTION

The inventors provide a series of methods for treating various diseases and conditions by employing reagents derived from genes whose expression is induced by hypoxia. In one embodiment, the invention provides a method of inhibiting angiogenesis associated with wound healing, retinopathy, ischemia, inflammation, microvasculopathy, bone healing, skin inflammation, or follicular development. An antisense polynucleotide comprising 15 or more consecutive nucleotides of the complement of a sequence selected from the group consisting of SEQ ID NO:1 (HOG3), SEQ ID NO:3 (HOG8), SEQ ID NO:5 (HOG18), SEQ ID NO:9 (CA9), SEQ ID NO:11 (HXB), SEQ ID NO:13 (IGFBP5), SEQ ID NO:15 (HFARP), SEQ ID NO: 17(STC1), SEQ ID NO: 19 (mig-6) and SEQ ID NO:21 (SSR4) is provided to a patient suffering from abnormalities of wound healing, retinopathy, ischemia, inflammation, microvasculopathy, bone healing, skin inflammation, or follicular development, following which angiogenesis is inhibited in the patient.

In another embodiment, the invention provides another method of inhibiting angiogenesis associated with wound healing, retinopathy, ischemia, inflammation, microvasculopathy, bone healing, skin inflammation, or follicular development. An antibody which specifically binds to a polypeptide selected from the group consisting of SEQ ID NO:2 (HOG3), SEQ ID NO:4 (HOG8), SEQ ID NO:6 (HOG18), SEQ ID NO: 10 (CA9), SEQ ID NO: 12 (HXB), SEQ ID NO: 14 (IGFBP5), SEQ ID NO: 16 (HFARP), SEQ ID NO:18 (STC1), SEQ ID NO:20 (mig-6) and SEQ ID NO:22 (SSR4) is administered to the patient, following which angiogenesis is inhibited in the patient.

Still another embodiment of the invention provides a method of promoting angiogenesis associated with wound healing, retinopathy, ischemia, inflammation, microvasculopathy, bone healing, skin inflammation, or follicular development. A polypeptide selected from the group consisting of SEQ ID NO:2 (HOG3), SEQ ID NO:4 (HOG8), SEQ ID NO:6 (HOG18), SEQ ID NO:10 (CA9), SEQ ID NO:12 (HXB), SEQ ID NO: 14 (IGFBP5), SEQ ID NO: 16 (HFARP), SEQ ID NO: 18 (STC1), SEQ ID NO:20 (mig-6) and SEQ ID NO:22 (SSR4) is administered to a patient, and angiogenesis is promoted in the patient.

Even another embodiment of the invention provides another method of promoting angiogenesis associated with wound healing, retinopathy, ischemia, inflammation, microvasculopathy, bone healing, skin inflammation, or follicular development. A vector comprising a nucleotide sequence encoding a polypeptide selected from the group consisting of SEQ ID NO:2 (HOG3), SEQ ID NO:4 (GOG8), SEQ ID NO:6 (HOG18), SEQ ID NO: 10 (CA9), SEQ ID NO: 12 (HXB), SEQ ID NO: 14 (IGFBP5), SEQ ID NO:16 (HFARP), SEQ ID NO: 18 (STC1), SEQ ID NO:20 (mig-6) and SEQ ID NO:22 (SSR4) and a promotor is administered to a patient. The nucleotide sequence is operably linked to the promoter and is transcribed into a sense mRNA upon transcription of the vector, whereupon angiogenesis is promoted in the patient.

In another embodiment the invention provides a method of treating a tumor. An antisense polynucleotide comprising 15 or more consecutive nucleotides of the complement of a sequence selected from the group consisting of SEQ ID NO: 1 (HOG3), SEQ ID NO:3 (HOG8), SEQ ID NO:5 (HOG 18), SEQ ID NO: 13 (IGFBP5), SEQ ID NO: 15 (HFARP), SEQ ID NO: 19 (mig-6) and SEQ ID NO:21 (SSR4) is administered to the patient and tumor growth is inhibited.

Yet another embodiment of the invention provides a method of treating a tumor, in which an antibody which specifically binds to a polypeptide selected from the group consisting of SEQ ID NO:2 (HOG3), SEQ ID NO:4 (HOG8), SEQ ID NO:6 (HOG28), SEQ ID NO:14 (IGFBP5), SEQ ID NO:16 (HFARP), SEQ ID NO:20 (mig-6) and SEQ ID NO:22 (SSR4) is admisistered to a patient. Tumor growth in the patient is inhibited.

Still another embodiment of the invention provides a method of diagnosing cancer in a subject. A polypeptide selected from the group consisting of SEQ ID NO:2 (HOG3), SEQ ID NO:4 (HOG8), SEQ ID NO:6 (HOG18), SEQ ID NO: 14 (IGFBP5), SEQ ID NO:16 (HFARP), SEQ ID NO:20 (mig-6) and SEQ ID NO:22 (SSR4) is quantified in a test sample suspected of being neoplastic from the subject and in a non-neoplastic control sample. The quantity of the polypeptide in the test sample is compared with the quantity of the polypeptide in the non-neoplastic control sample. The subject is identified as having a cancer if the quantity of the protein is higher in the test sample than in the control sample.

Yet another embodiment of the invention provides a method of diagnosing cancer in a subject. An mRNA selected from the group consisting of SEQ ID NO:1 (HOG3), SEQ ID NO:3 (HOG8), SEQ ID NO:5 (HOG18), SEQ ID NO:13 (IGFBP5), SEQ ID NO:15 (HFARP), SEQ ID NO:19 (mig-6) and SEQ ID NO:21 (SSR4) is quantified in a test sample suspected of being neoplastic from the subject and in a non-neoplastic control sample. The quantity of the mRNA in the test sample is compared with the quantity of the mRNA in the non-neoplastic control sample. The subject is identified as having a cancer if the quantity of the protein is higher in the test sample than in the control sample.

Another embodiment provides a method of imaging a tumor. An antibody which specifically binds to a polypeptide selected from the group consisting of SEQ ID NO:2 (HOG3), SEQ ID NO:4 (HOG8), SEQ ID NO:6 (HOG18), SEQ ID NO:8 (PLOD2), SEQ ID NO:14 (IGFBP5), SEQ ID NO:16 (HFARP), SEQ ID NO:20 (mig-6) and SEQ ID NO:22 (SSR4) is administered to a subject or to a tissue sample from a subject. The antibody is covalently linked to a label. The label is detected and an image is formed of the distribution of the label in the subject or tissue sample.

### BRIEF DESCRIPTION OF THE DRAWINGS

The application file contains at least one drawing executed in color. Copies of this patent application publication with color drawings will be provided by the Office upon request and payment of the necessary fee.

FIGS. 1A and 1B show the time course of expression of HOGs in 1.5% oxygen. In FIG. 1A cultured glioblastoma cells (D247-MG) were switched to 1.5% oxygen at zero hours and the levels of transcripts of the individual genes determined by real-time PCR to produce the time-course of the hypoxia response. In FIG. 1B the time course of CA9 protein expression was measured by western blot analysis of lysates from D247-MG cells grown in atmospheric oxygen or 1.5% oxygen. Molecular weight markers are shown to the left.

FIGS. 2A and 2B show HOG induction by HIF-1 or hypoxia in malignant cells. In FIG. 2A D247-MG cells were transfected with HIF-1α and cultured at either atmospheric or 1% oxygen. Transcript levels are displayed relative to the same standard as determined by real-time PCR. FIG. 2B depicts HOG induction in malignant cell lines derived from commonly occurring cancers as determined by lowering the oxygen concentration from atmospheric to 1.5% oxygen and measuring induction by real-time PCR. The cell lines used were Normal Human Astrocytes (1); glioblastomas D263-MG (2), D392-MG (3), D502-MG (4), D566-MG (5) and U87 (6); medulloblastomas D283-Med (7), D341-Med (8), D425-Med (9), D556-Med (10), D581-Med (11) and UW228 (12); colon carcinomas SW480 (13) and HCT116 (14); non-small lung carcinoma NCI-H23 (15); and breast cancers SKBr3 (16) and MCF7 (17). Genes induced greater than 10-fold are displayed as 10-fold.

FIGS. 3A-3N show in vivo expression of HOGs in human solid tumors. Immunohistochemistry was used to co-localize CA9 (FIG. 3A, brown stain) and the chemical hypoxia marker, pimonidazole (FIG. B, green stain) in serial sections of an oropharyngeal squamous cell carcinoma, sccNij70. Regions staining red in B represents proliferating (IdUrd labeled) cells. A standard H & E stain of an adjacent section (FIG. 3C) was used to show necrotic cells (staining red). In situ hybridization for NDRG1 transcript (FIG. 3E) shows co-localization with CA9 (FIG. 3D) and pimonidazole (FIG. 3F) in an oropharyngeal squamous cell carcinoma. Peri-necrotic staining in GBMs was observed for CA9 (FIGS. 3G and 3J), BNIP3 (FIGS. 3H and 3K), NDRG1 (FIGS. 31 and 3L), IGFBP3 (FIG. 3M) and HFARP (FIG. 30). IGFBP3 stains endothelial cells in addition to hypoxic regions not adjacent to vessels (FIG. 3N). Arrows point to necrotic areas. Magnification was 10× for 3A to 3C, 3G to 31 and 3M; 25× for 3D to 3F, 3L and 3O; 50× for 3J and 3K; and 100× for 3N.

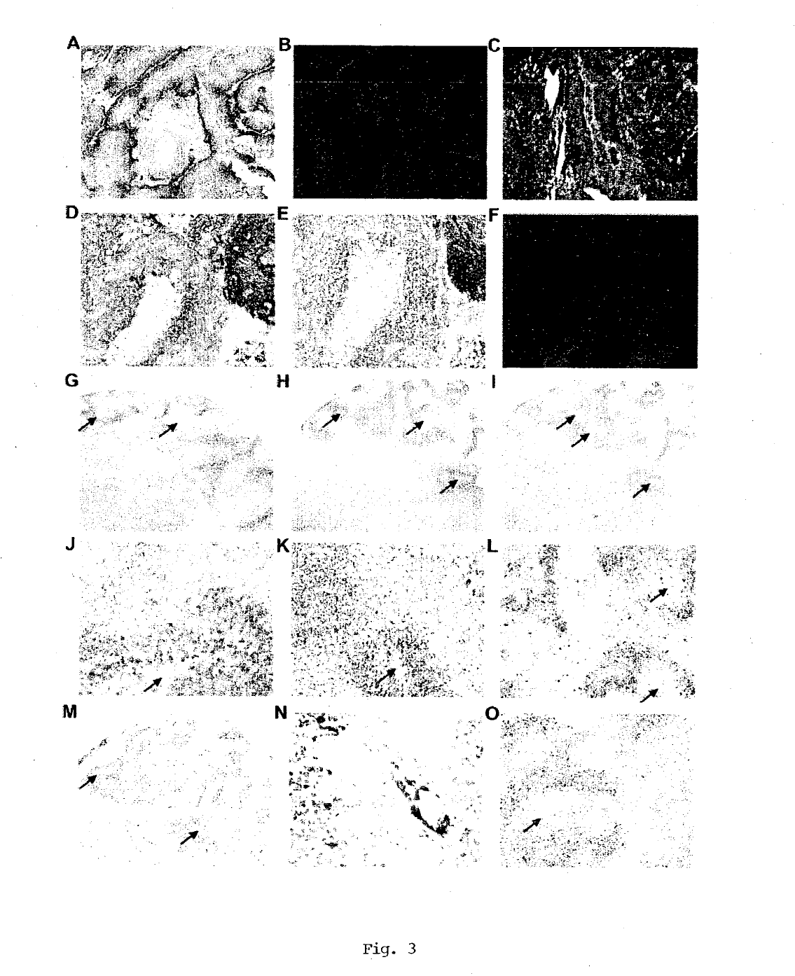

### DETAILED DESCRIPTION OF THE INVENTION

The present inventors have discovered that the expression of certain genes is elevated in cells grown under hypoxic conditions. Specifically, the inventors discovered that expression of the genes HOG18, HOG3, HOG8, PLOD2, CA9, HXB, IGFBP5, STC1, HFARP, mig-6, and SSR4 is increased under hypoxic conditions in human glioblastoma cells and several human tumors in situ. These and other hypoxia overexpressed genes (HOGs) can be used for the diagnosis and treatment of cancer and angiogenesis-related conditions. The practical applications of the discovery include the use of antisense polynucleotides and antibodies as antitumor agents, the use of antisense polynucleotides and antibodies to disrupt angiogenesis in pathological tissues, the use of polynucleotides or polypeptides to promote angiogenesis in wound healing or regeneration of tissues, and the use of oligonucleotide probes and antibodies as tumor markers in diagnosis and prognosis.

HOGs were identified based on Serial Analysis of Gene Expression (SAGE) (Velculescu V E, et al., Science 270:484-87 (1995)) of cells cultured under low oxygen conditions. Eleven genes (HOG18, HOG3, HOG8, PLOD2, CA9, HXB, IGFBP5, STC1, HFARP, mig-6, and SSR4) were identified whose expression previously was not known to be induced by hypoxia. Full-length cDNA sequences of these genes have been previously reported. The cDNA sequences for HOG18, HOG3, HOG8, PLOD2, CA9, HXB, IGFBP5, STC1, HFARP, mig-6, and SSR4 are shown in SEQ ID NOS:1, 3, 5, 7, 9, 11, 13, 15, 17, 19, and 21, respectively, and the corresponding encoded amino acid sequences are shown in SEQ ID NOS:2, 4, 6, 8, 10, 12, 14, 16, 18, 20, and 22. No function has been assigned previously to HOG18 (accession number NM_{—}019058), HOG3 (accession number NM_{—}017606), and HOG8 (accession number BC007832). Known functional properties of the remaining eight genes are summarized below.

The angiopoietin-related gene, HFARP, encodes a secreted protein reported to protect endothelial cells from apoptosis (Kim et al., Biochem J 346 Pt 3:603-610 (2000)).

Transcription of mig-6 is induced by glucocorticoids, insulin, cAMP, retinoic acid vasoactive peptides, serun mitogen, diabetic nephropathy, and stress (Lee et al., Arch Biochem Biophys 269:106-113 (1989); Kent et al., Endocrinology 134:2237-2344 (1994); Wick et al. (Exp Cell Res 219(2):527-535 (1995); Makkinje et al., J Biol Chem 275:17838-47 (2000)). Transcription of mig-6 is regulated during the cell cycle, with peak levels around mid G1 (Varley et al., Biochem Biophys Res Commun 254:728-733 (1999)).

The delta subunit of signal sequence receptor, also referred to as SSR4 or translocon-associated protein (TRAP) delta, spans the ER membrane once and has most of its mass at the lumenal side (Hartmann et al., Eur J Biochem 214(2):375-381 (1993)). The genomic and cDNA of human SSR4 has been isolated (Brenner et al., Genomics 44(1):8-14 (1997)).

CA9 is expressed in renal cell and cervical carcinomas and is being exploited for diagnostic (Uemura et al., Br J Cancer 81(4):741-746 (1999); Nogradi A, Am J Pathol 154:1-1 (1998); Vermylen et al., Eur Respir J 14(4):806-811 (1999); U.S. Pat. No. 6,087,09) and therapeutic (Zavada et al, Br J Cancer 82(11):1808-1813 (2000)) U.S. Pat. No. 5,387,676) purposes. CA9 is regulated by vHL in renal cells through degradation of HIF-1α. CA9 is also increased by vHL mutations and shows peri-necrotic staining in various tumors (Ivanov et al., Proc Natl Acad Sci USA 95:12596-12601 (1998); Wykoff C C, et al. Cancer Res 60:7075-83 (2000)).

Hexabrachion (HXB) is an extracellular matrix glycoprotein which promotes endothelial cell sprouting with basic fibroblast growth factor (Schenk S, Mol Biol Cell 10:2933-43 (1999)). Expression of HXB is correlated with angiogenesis in breast cancer, gliomas, and lymphomas (Vacca A, et al., Leuk Lymphoma 22:473-81 (1996); Jallo G I, et al., Neurosurgery 41:1052-9 (1997); Tokes A M, et al., Pathol Res Pract 195:821-8 (1999)). Antibodies specific for HXB can inhibit angiogenesis (Canfield A E and Schor A M, J Cell Sci 108:797-809 (1995)) and anti-sense therapy halts vascular thickening of pulmonary arteries (Cowan K N, et al., J Clin Invest 105:21-34 (2000)). Bigner & Zalutsky (U.S. Pat. No. 5,624,659) have described methods of treating brain tumors using radiolabeled monoclonal antibodies to HXB. Kimura (U.S. Pat. No. 5,436,132) has demonstrated the quantitative determination of HXB by immunoassay in cerebrospinal fluid as a glioma marker.

PLOD2 is a lysyl hydroxylase which is involved in angiogenesis. Inhibitors of PLOD2 block collagen synthesis and promote the effectiveness of other compounds which inhibit angiogenesis (U.S. Pat. No. 5,021,404). PLOD2 acts synergistically to inhibit angiogenesis when administered together with an angiostatic compound such as heparin or a heparin analogue (U.S. Pat. No. 5,021,404). Several inhibitors of PLOD2 are known (U.S. Pat. Nos. 5,328,913 and 4,797,471).

Ischemia produces an immediate decrease in expression of IGFBP5 in neonatal rat brain (Clawson et al., Biol Signals Recept 8(4-5):281-293 (1999)). At longer times following an ischemic event, stimulation of IGFBP5 expression has been observed (Lee, et al. J Cereb Blood Flow Metab 16(2):227-236 (1996); Clawson et al., Biol Signals Recept 8(4-5):281-293 (1999)).

IGFBP5 and stanniocalcin (STC1) have been used as markers for vascular endothelial cells in tumors (St. Croix B, et al. Science 289:1197-202 (2000)). STC1 is induced during endothelial cell differentiation in an in vitro model (Kahn J, et al. Am J Pathol 156:1887-900 (2000)). STC1 mRNA is found in several cancer cell lines and tumor tissues, and the use of STC1 as a molecular marker for tumors has been suggested (Fujiwara et al., Int J Oncol 16:799-804 (2000); Miura W, et al., APMIS 108:367-372 (2000)).

Disrupting the expression of any one of HOG3, HOG8, HOG18, PLOD2, HFARP, mig-6, CA9, HXB, SSR4, IGFBP5, and STC1 individually or in combination can be used to inhibit or treat angiogenesis-related conditions. Such conditions include retinopathy, microvasculopathy, inflammatory conditions such as rheumatoid arthritis, and skin inflammations like psoriasis. Antisense oligonucleotides or antisense polynucleotides that specifically bind to transcripts of these genes can be used to prevent their translation in vivo.

Oligonucleotides or polynucleotides based on the genes identified here can be delivered therapeutically to cells to inhibit angiogenesis. As defined herein, the terms “oligonucleotide” and “polynucleotide” are used interchangeably and either refers to two or more nucleotides linked covalently through phosphodiester bonds. Antisense constructs of HOG3, HOG8, HOG18, PLOD2, HFARP, mig-6,CA9, HXB, SSR4, IGFBP5 or STC1, either alone or in combination, can be administered therapeutically to inhibit angiogenesis. Antisense constructs typically contain a promoter located 3′ to and operably linked to the sequence encoding the desired antisense polynucleotide or antisense polynucleotide. Upon initiation of transcription at the promoter, an RNA molecule is transcribed which is complementary to the native mRNA molecule of the gene.

The polynucleotides of the present invention encode all or a portion of the polypeptides HOG3, HOG8, HOG18, PLOD2, HFARP, mig-6,CA9, HXB, SSR4, IGFBP5 and STC1. These polynucleotides can be isolated and purified free from other nucleotide sequences by standard purification techniques, using restriction enzymes to isolate fragments comprising the coding sequences of interest. The polynucleotide molecules are preferably intron-free. Such cDNA molecules can be made inter alia by using reverse transcriptase with HOG3, HOG8, HOG18, HFARP, mig-6,CA9, HXB, SSR4, IGFBP5 and STC1 mRNA as a template. The polynucleotide molecules of the invention can also be made using the techniques of synthetic chemistry. The degeneracy of the genetic code permits alternate nucleotide sequences to be synthesized that will encode the desired amino acid sequence. All such nucleotide sequences are within the scope of the present invention. Degenerate nucleotide sequences encoding the polypeptides HOG3, HOG8, HOG18, PLOD2, HFARP, mig-6,CA9, HXB, SSR4, IGFBP5 and STC1, as well as homologous nucleotide sequences which are at least about 50, 55, 60, 65, 70, preferably about 75, 90, 96, or 98% identical to a nucleotide sequence shown in SEQ ID NOS:1, 3, 5, 7, 9, 11, 13, 15, 17, 19, and 21 and the complements thereof also are within the scope of the present invention. Percent sequence identity between the sequences of two polynucleotides is determined using computer programs such as ALIGN which employ the FASTA algorithm, using an affine gap search with a gap open penalty of −12 and a gap extension penalty of −2. Complementary DNA (cDNA) molecules, species homologs, and variants of HOG3, HOG8, HOG18, PLOD2, HFARP, mig-6,CA9, HXB, SSR4, IGFBP5 and STC1 which encode polypeptides with comparable biological activity also are within the scope of the present invention. Polynucleotide molecules of the invention can be propagated in vectors and cell lines as is known in the art. The constructs may be on linear or circular molecules. They may be on autonomously replicating molecules or on molecules without replication sequences.

Any technique available in the art can be used to introduce genetic constructs into the cells. These include, but are not limited to, transfection with naked or encapsulated nucleic acids, cellular fusion, protoplast fusion, viral infection, and electroporation. Introduction of genetic constructs may be carried out in vitro or in vivo.

Antisense intervention in the expression of specific genes can also be achieved by the use of synthetic antisense polynucleotide sequences (see Lefebvre-d'Hellencourt et al, Eur Cytokine Net. 6:7 (1995); Agrawal, Tibtech, 14:376 (1996); Lev-Lehman et al, Antisense Oligomers in vitro and in vivo. In Antisense Therapeutics, A. Cohen and S. Smicek, eds (Plenum Press, New York) (1997)). Antisense polynucleotide sequences may be short sequences of DNA, typically at least 12, 15, 17, 20, 25, 30, 35, 40, 45, or 50 nucleotides in length, but may be as small as a 7-mer (Wagner et al, Nature Biotechnology 14:840-844 (1996)), designed to complement a target mRNA of interest and form an RNA:antisense duplex. This duplex formation can prevent processing, splicing, transport or translation of the relevant mRNA. An antisense compound hybridizes specifically when binding of the compound to the target RNA molecule interferes with the normal function of the target RNA and there is little or no measurable non-specific binding of the antisense compound to non-target sequences under conditions used for assays or in vivo therapeutic treatment.

When employed as pharmaceuticals, the antisense polynucleotides are usually administered in the form of pharmaceutical compositions. These compounds can be administered by a variety of routes including oral, rectal, transdermal, subcutaneous, intraperitoneal, intravenous, intramuscular, and intranasal. Pharmaceutical compositions containing oligonucleotides of the invention are prepared in any manner well known in the pharmaceutical art and comprise at least one active compound. It is contemplated that the pharmaceutical composition can be administered directly into a tumor to be treated. The compositions of the invention can be formulated so as to provide quick, sustained, or delayed release of the active ingredient after administration to the patient by employing formulations known in the art.

Another method of delivery involves delivery of the naked antisense polynucleotides across the dermal layer. The delivery of naked antisense polynucleotides is well known in the art. See, for example, Feigner et al., U.S. Pat. No. 5,580,859. It is contemplated that the antisense polynucleotides can be packaged in a lipid vesicle before delivery of the antisense polynucleotide.

An antisense polynucleotide or antisense construct is effective over a wide dosage range and is generally administered in a pharmaceutically effective amount. An effective amount is that amount which when administered alleviates the symptoms or inhibits tumor cell growth. Normal dosage amounts can vary from 0.1 to 100,000 micrograms, up to a total dose of about 1 g, depending upon the route of administration. The course of therapy may last minutes, hours, days, or up to several months or until diminution of the disease is achieved. Preferably the effective amount is from about 0.02 mg/kg body weight to about 20 mg/kg body weight. However, the amount of the antisense polynucleotide or antisense construct actually administered usually will be determined by a physician in light of the relevant circumstances, including the condition to be treated, the chosen route of administration, the actual compound administered, the age, weight, and response of the individual patient, the severity of the patient's symptoms, and the like.

Antibodies or antigen-binding fragments that bind to any one of the polypeptides HOG3, HOG8, HOG18, PLOD2, HFARP, mig-6, CA9, HXB, SSR4, IGFBP5, or STC1 can be used individually or in combination to inhibit angiogenesis. Antibodies or antigen-binding fragments bind specifically to these gene products preventing physiological action of the polypeptides.

Antibodies directed against the polypeptides of this invention are immunoglobulins (e.g., IgG, IgA, IgM, IgD, or IgE) or portions thereof that are immunologically reactive with the polypeptide of the present invention. As used herein, the term “antibody” includes whole immunoglobulin molecules, fragments of immunoglobulin molecules, and modified or synthetic immunoglobulins. The term “antibody” also includes single-chain antibodies, which generally consist of a variable domain of a heavy chain linked to a variable domain of a light chain. The production of single-chain antibodies is well known in the art (see, e.g., U.S. Pat. No. 5,359,046). An antibody of this invention may also be a humanized antibody, which refers to a molecule that has its antigen-binding regions derived from a non-human species immunoglobulin and the remainder of the antibody molecule derived mainly from a human immunoglobulin antibodies which are known in the art (see, e.g. U.S. Pat. Nos. 5,777,085 and 5,789,554). It can be a molecule that has multiple binding specificities, such as a bifunctional antibody. Bifunctional antibodies can be prepared by any technique known to those of skill in the art, including the production of hybrid hybridomas, disulfide exchange, chemical cross-linking, addition of peptide linkers between two monoclonal antibodies, the introduction of two sets of immunoglobulin heavy and light chains into a particular cell line, and so forth. Alternatively, peptides corresponding to specific regions of the polypeptide encoded by the target gene may be synthesized and used to create immunological reagents according to well known methods.

Antibodies directed against a polypeptide encoded by a target gene may be generated by immunization of a mammalian host, including a rat, rabbit, goat, sheep, horse, pig, or primate. Such antibodies may be polyclonal or monoclonal. Preferably they are monoclonal. Methods to produce polyclonal and monoclonal antibodies are well known to those of skill in the art. For a review of such methods, see Harlow & Lane (1988) Antibodies, A Laboratory Manual; Yelton, et al., Ann. Rev. of Biochem. 50:657-80 (1981); and Ausubel, et al., Current Protocols in Molecular Biology, John Wiley & Sons, (1989)). Determination of immunoreactivity with a polypeptide encoded by a target gene may be made by any of several methods well known in the art, including by immunoblot assay and ELISA. Monoclonal antibodies with affinities of 10⁻⁸ M⁻¹ or preferably 10⁻⁹ to 10⁻¹⁰ M⁻¹ or stronger are considered specific to a given protein and are typically made by standard procedures as described, e.g., in Harlow & Lane, 1988.

Additionally, one of skill in the art has a variety of methods available which may be used to alter the biological properties of the antibodies of this invention. Such methods include chemical alteration, addition of buffer components, or amino acid substitutions which can increase or decrease the stability or half-life, immunogenicity, toxicity, affinity, or yield of a given antibody molecule.

Angiogenesis can also be inhibited by decreasing translation of mRNA by reducing the amount of available mRNA through the use of ribozymes that are capable of cleaving mRNA expressed by HOG3, HOG8, HOG18, PLOD2, HFARP, mig-6, CA9, HXB, SSR4, IGFBP5, and STC1. Ribozymes can be administered directly or as a ribozyme-expressing construct. The primary sequence of the target gene can be used to design ribozymes that can target and cleave specific essential gene sequences. There are a number of different types of ribozymes. Most synthetic ribozymes are generally hammerhead, Tetrahymena, and hairpin ribozymes. Methods of designing and using ribozymes to cleave specific RNA species are known in the art, see Zhao, et al., Mol Cell Neurosci 11:92-97(1998); Lavrovsky et al. (1997); and Eckstein “Exogenous Application of Ribozymes for Inhibiting Gene Expression”, in Oligonucleotides as Therapeutic Agents, Ciba Foundation Symposium 209, John Wiley & Sons, Chichester, England, pp. 207-217 (1997)).

It is sometimes desirable to promote angiogenesis, for example, to aid in wound healing, bone healing, follicular development, tissue regeneration following ischemia, or other conditions in which increased blood flow to a tissue or organ is desirable. Increased vascularization results in increased blood flow, which aids in healing and developing damaged tissues. Angiogenesis can be promoted by administering any one of the polypepetides HOG3, HOG8, HOG18, PLOD2, HFARP, CA9, HXB, mig-6, SSR4, IGFBP5, or STC1, individually or in combination. Methods of polypeptide expression, purification, and formulation are well-known in the art and any may be used without limitation.

It is also possible to increase expression of HOG3, HOG8, HOG18, PLOD2, HFARP, CA9, HXB, mig-6, SSR4, IGFBP5, or STC1 by administering a vector comprising at least seven nucleotides that encode any part or all of one or more of the genes HOG3, HOG8, HOG18, PLOD2, HFARP, CA9, HXB, mig-6, SSR4, IGFBP5, or STC1 operably linked to a promoter. Expression of sense mRNA molecules encoding HOG3, HOG8, HOG18, HFARP, mig-6,CA9, HXB, SSR4, IGFBP5 or STC1 polypeptides promotes angiogenesis. Methods for obtaining the polynucleotides required for this embodiment are well-known in the art.

Disrupting the expression of any one of HOG3, HOG8, HOG18, PLOD2, HFARP, mig-6, SSR4, and IGFBP5 individually or in combination can be used to treat tumors. These genes are important in vascularization of tumors, because vascularization allows tumors to increase in size and to undergo metastasis. Antisense polynucleotides or oligonucleotides targeted to these genes can be used to prevent translation in vivo, which can prevent angiogenesis and stop or reduce tumor growth. Gene therapy to increase expression of angiostatin, an inhibitor of angiogenesis, was recently demonstrated to inhibit the growth of tumors in mice (Matsumoto et al., Oral Oncol 37:369-78 (2001)), thereby establishing the feasibility of blocking tumor growth by introducing genes which inhibit angiogenesis. Production and use of antisense polynucleotides is known in the art and was discussed previously. Antibodies or antigen-binding fragments that bind to one of the polypeptides HOG3, HOG8, HOG18, HFARP, mig-6, SSR4, or IGFBP5 can also be used individually or in combination to treat tumors.

Quantifying gene expression of HOG3, HOG8, HOG18, HFARP, mig-6, PLOD2, SSR4, or IGFBP5, either singly or in combination, can be used to diagnose cancer in a subject. Expression of HOG3, HOG8, HOG18, HFARP, mig-6, PLOD2, SSR4, or IGFBP5 in a test sample suspected of being cancerous can be compared to the expression of the same gene or genes in a second sample from a normal subject. Increased expression of at least one gene in the test sample relative to the normal sample identifies the test sample as potentially cancerous. Any method for observing gene expression can be used, without limitation. Common methods are quantification of expressed mRNA, e.g., by Northern blot analysis or other hybridization techniques, or quantification of expressed polypeptides by SDS-PAGE, Western blot, or immunoassay.

For gene therapy purposes, cells can be transfected in vitro and administered to a subject. Alternatively, cells can be directly transfected in vivo. Delivery of nucleic acid molecules can be accomplished by any means known in the art. Gene delivery vehicles are available for delivery of polynucleotides to a cell, a tissue, an organ, or a mammal for expression. For example, a polynucleotide or oligonucleotide of the invention can be administered either locally or systemically in a gene delivery vehicle. Gene delivery constructs can contain viral or non-viral vectors in either in vivo or ex vivo modality. Expression of the gene of interest can be driven by endogenous mammalian or heterologous promoters. Expression of the coding sequence in vivo can be either constitutive or regulated. The invention includes gene delivery vehicles capable of expressing the contemplated polynucleotides. The gene delivery vehicle is preferably a viral vector and, more preferably, a retroviral, adenoviral, adeno-associated viral, herpes viral, or alphaviral vector. The viral vector can also be an astroviral, coronaviral, orthomyxoviral, papovaviral, paramyxoviral, parvoviral, picomaviral, poxviral, togaviral vector. See generally, Jolly, Cancer Gene Therapy 1:51-64 (1994); Kimura, Human Gene Therapy 5:845-852 (1994), Connelly, Human Gene Therapy 6:185-193 (1995), and Kaplitt, Nature Genetics 6:148-153 (1994).

Delivery of the gene therapy constructs of this invention into cells is not limited to the above mentioned viral vectors. Other delivery methods may be employed such as, for example, nucleic acid expression vectors; polycationic condensed DNA (see Curiel, Hum Gene Ther 3:147-154 (1992); ligand linked DNA (see Wu, J. Biol. Chem. 264:16985-16987 (1989)); eucaryotic cell delivery vehicles (see U.S. Pat. No. 6,015,686); deposition of photopolymerized hydrogel materials; hand-held gene transfer particle gun (U.S. Pat. No. 5,149,655); ionizing radiation (U.S. Pat. No. 5,206,152 and WO 92/11033); nucleic charge neutralization; or fusion with cell membranes. Additional approaches are described in Philip, Mol. Cell. Biol. 14:2411-2418 (1994) and in Woffendin, Proc. Natl. Acad. Sci. 91:1581-585 (1994). The sequence can be inserted into a vector containing control sequences for high level expression. The vector can be incubated with synthetic gene transfer molecules including polymeric DNA-binding cations like polylysine, protamine, or albumin. A DNA-binding molecule can in turn be linked, preferably covalently, to a cell targeting ligand which binds specifically to a desired cell surface receptor expressed on a target cell. Targeting ligands include, for example, asialoorosomucoid (Wu and Wu, J. Biol. Chem. 262:4429-4432 (1987)); insulin (Hucked, Biochem. Pharmacol. 40:253-263 (1990)); galactose (Plank, Bioconjugate Chem 3:533-539 (1992)); lactose; and transferrin. Naked DNA may also be employed. Exemplary naked DNA introduction methods are described in PCT Patent Publication No. WO 90/11092 and U.S. Pat. No. 5,580,859. Uptake efficiency may be improved using biodegradable latex beads which are efficiently transported into cells after endocytosis. The method may be improved further by treatment of the beads to increase hydrophobicity and thereby facilitate disruption of the endosome and release of the DNA into the cytoplasm. Liposomes, that can act as gene delivery vehicles are described in U.S. Pat. No. 5,422,120, PCT Patent Publication Nos. WO 95/13796, WO 94/23697, and WO 91/144445, and EP No. 524,968.

The pharmaceutical compositions of this invention may be administered using microspheres, microparticulate delivery systems, or other sustained release formulations. Sustained release formulations can be placed in, near, or otherwise in communication with affected tissues or the bloodstream.

The methods of this invention also may be accomplished using liposomes, which can optionally contain other agents to aid in targeting or administration of the compositions to the desired treatment site. Liposomes containing compositions contemplated for use with methods of the invention may be prepared by well-known methods (See, e.g. DE 3,218,121; Epstein et al. (1985) Proc. Natl. Acad. Sci. U.S.A. 82:3688-92; Hwang et al. (1980) Proc. Natl. Acad. Sci. U.S.A. 77:4030-34; U.S. Pat. Nos. 4,485,045 and 4,544,545).

Effective doses of the pharmaceutical compositions of the present invention will vary depending upon many different factors, including the form of the composition administered, the means of administration, target site, physiological state of the patient, antibody affinity, and other medicments administered. Thus, treatment dosages will need to be titrated to optimize safety and efficacy; such can be readily determined and are routine to the ordinarily skilled artisan. In determining the effective amount of polypeptide or polynucleotide to be administered, the physician evaluates, for example, the particular composition used, the disease state being diagnosed; the age, weight, and condition of the patient, formulation toxicities, disease progression, etc. The dose will also be determined by the existence, nature, and extent of any adverse side-effects that accompany the administration of a particular formulation. Doses ranging from about 10 ng to 1 g polypeptide per patient are typical. Doses generally range between about 0.01 and about 50 mg polypeptide per kilogram of body weight; preferably between about 0.1 and about 5 mg/kg polypeptide of body weight.

Oligonucleotide probes and antibodies can be used as tumor markers in diagnosis and prognosis of cancer. The expression product monitored may be RNA or protein. Multiple expression products, e.g., 2, 3, 4, 5, 7, 10, 15, 20, 30, 50, 100, 300, 500, or 1000 or more expression products can be quantified simultaneously. Methods of monitoring gene expression are well known in the art and any may be used. For example, RNA levels can be measured by Northern blotting and other hybridization techniques, nuclease protection, microarrays, RT-PCR, and differential display. The term quantifying when used in the context of quantifying transcription levels of a gene can refer to absolute or to relative quantification. Absolute quantification may be accomplished by inclusion of known concentration(s) of one or more control target nucleic acids and referencing the hybridization intensity of unknowns with the known target nucleic acids, e.g., through generation of a standard curve. Alternatively, relative quantification can be accomplished by comparison of hybridization signals between a sample derived from a test subject and a sample derived from a normal subject to determine differences in hybridization intensity and, by implication, transcription level.

One of skill in the art can readily determine differences in the amount of gene expression product from the test sample as compared to a normal subject using, e.g., Northern blots and nucleotide probes. The quantity of mRNA expressed from at least one of HOG3, HOG8, HOG18, HFARP, mig-6, PLOD2, SSR4, or IGFBP5 in a test sample of a human suspected of having cancer, can be compared with the mRNA expression from at least one of HOG3, HOG8, HOG 18, HFARP, mig-6, PLOD2, SSR4, and IGFBP5 in a normal sample. This can be done, for example, using in situ hybridization in tissue section or in Northern blots containing mRNA. A higher level of mRNA expressed from a gene represented by a HOG3, HOG8, HOG108, HFARP, mig-6, PLOD2, SSR4, and IGFBP5 polynucleotide in the test sample as compared to the normal sample is indicative or suggestive of cancer in the suspect human who has provided the test sample. Preferably, the increased level of mRNA expressed from a HOG3, HOG8, HOG18, HFARP, mig-6, PLOD2, SSR4, or IGFBP5 gene in the test sample is at least 25%, 50%, 100%, 150%, 200%, or 250% higher than in the normal body sample.

To facilitate detection any polynucleotide or oligonucleotide of this invention can be labeled using standard methods. A wide variety of labels and conjugation techniques are known and are reported extensively in both the scientific and patent literature. For example, polynucleotides or oligonucleotides can be radiolabeled with ³²P or covalently linked to a fluorescent or biotinylated molecule. Other techniques such as high density DNA array hybridization, ribonuclease protection assay, and serial analysis of gene expression can also be used. Oligonucleotide probes specific to the nucleotides encoded by HOG3, HOG8, HOG18, HFARP, mig-6, PLOD2, SSR4, and IGFBP5 can be generated using the polynucleotide sequences of HOG3, HOG8, HOG18, HFARP, mig-6, PLOD2, SSR4, and IGFBP5 genes. The probes are preferably at least 12, 14, 16, 18, 20, 22, 24, or 25 nucleotides in length and can be less than 2, 1, 0.5, 0.1, or 0.05 kb in length. The probes can be, for example, synthesized chemically, generated from longer polynucleotides using restriction enzymes, or amplified enzymatically. The probes can be labeled, for example, with a radioactive, biotinylated, or fluorescent tag. A mixture of probes can also be used. Such mixture can contain a plurality of probes which are specific to different genes identified in this invention so that the expression of one or more genes can be monitored simultaneously. Alternatively, each of a plurality of probes can be used separately.

The antibodies of the present invention can be used to detect any one of HOG3, HOG8, HOG18, HFARP, mig-6, PLOD2, SSR4, and IGFBP5 in histological sections of glioma tissue as well as in other solid tumors, such as breast cancer and lung cancer. Tissue samples are preferably permeabilized with a sufficient amount of a suitable detergent to release membrane proteins into solution prior to immunological detection. One can detect antibody binding to extracts of tissue samples by any detection means known in the art, for example, radioimmunoassay, enzyme-linked immunoadsorbent assay, complement fixation, nephelometric assay, immunodiffusion, or immunoelectrophoretic assay. Alternatively, the antibodies can be used as an immunohistochemical reagents to visualize HOG3, HOG8, HOG18, HFARP, mig-6, PLOD2, SSR4, and IGFBP5 polypeptides in tissue sections.

A particularly useful stain for use in enzyme-linked antibody assays employs peroxidase, hydrogen peroxide and a chromogenic substance such as aminoethyl carbazole. The peroxidase (a well known enzyme available from many sources) can be coupled to an antibody specific for one of HOG3, HOG8, HOG 18, HFARP, mig-6, PLOD2, SSR4, or IGFBP5 or merely complexed to it via one or more antibodies. For example, a goat anti-peroxidase antibody and a goat antibody specific for one of HOG3, HOG8, HOGI 8, HFARP, mig-6, PLOD2, SSR4, and IGFBP5 can be complexed via an anti-goat IgG. Such techniques are well known in the art. Other chromogenic substances and enzymes may also be used.

The antibodies of the invention can be administered to a patient or to a tissue sample from a patient for locating a tumor or imaging analysis or a tumor. For such purposes, the antibodies are typically conjugated to an imaging agent, such as ¹²³I, ¹³¹I, or ¹¹¹In. Alternatively, ¹³C-enriched antibodies can also used in combination with magnetic resonance imaging. For in vitro analysis of tissue samples from a patient, a variety of imaging agents and techniques are known in the art. For example, the imaging agent can be a colored or fluorescent dye or an enzyme yielding a colored or fluorescent product. Methods of conjugation and production of isotopically enriched antibodies are routine and well known in the art. A diagnostically effective amount of antibody is one which allows the observer to distinguish between normal tissues and those containing elevated levels of HOG3, HOG8, HOG18, HFARP, mig-6, PLOD2, SSR4, or IGFBP5. Determination of such amounts is within the skill of the art. Methods of imaging or detecting the bound antibodies in a patient or in a tissue sample from a patient are also known in the art. For example, the patient may be scanned for radiation emitted by the imaging agent or a tissue section stained with the labeled antibody may be observed using a microscope.

The compounds of this invention can also be utilized in radioimmuno- or radiation therapy. This process differs from the corresponding diagnostic techniques only in the quantity and type of isotope employed. The objective is the destruction of tumor cells by high-energy shortwave radiation with a minimum range. Suitable β-emitting ions are, for example, ⁴⁶Sc, ⁴⁷Sc, ⁴⁸Sc, ⁷²Ga, ⁷³Ga and ⁹⁰Y. Suitable α-emitting ions exhibiting short half-life periods are, for example, ²¹¹Bi, ²¹²Bi, ²¹³Bi and ²¹⁴Bi. A suitable nuclide emitting photons and elections is ¹⁵⁸Gd which can be obtained from ¹⁵⁷Gd by neutron capture.

All references and patents cited herein are incorporated by reference in their entirety.

The following examples are provided by way of illustration and are not intended to limit the scope of the invention in any way.

### EXAMPLES

### Example 1

### Comparison of Gene Expression in Normal and Hypoxic Human Glioblastoma Cells

One application of the present invention involves quantitative comparison of gene expression in normal and tumor cells. RNA expression levels of HOG3, HOG8, HOG18, HFARP, mig-6, PLOD2, SSR4, and IGFBP5 were compared in normal and hypoxic human glioblastoma cells.

Cells were grown using standard cell culture techniques either in equilibrium with atmospheric oxygen or using 1.5% oxygen, approximating tumor hypoxia levels. Real-time PCR from a cDNA template was performed using a thermocycler with continuous fluorescent monitoring capabilities (LightCycler™, Roche Diagnostics) and SYBR Green I (Molecular Probes, Eugene, Oreg.) to analyze the kinetics of PCR product accumulation. PCR conditions and data analysis were reproduced as described (Loging W T, et al., Genome Res 10:1393402 (2000)) except 0.5 μM PCR primer and 500 μM of each dNTP was used. Primers specific for a 221-bp segment of β-actin were used to confirm cDNA integrity and normalization of cDNA yields. Primers specific for each hypoxia-inducible gene were designed with 140- to 240-bp products (all primer sequences available upon request). Relative expression levels were determined in duplicate by comparison to a serially diluted standard using the thermocycler software.

Measurement of transcript levels using real-time PCR and SAGE analysis showed that expression of HOG3, HOG8, HOG18, HFARP, mig-6, PLOD2, SSR4, and IGFBP5 in hypoxic glioblastoma cells was increased between 2- and 12-fold as compared to cells grown under normal aerobic conditions (Table 1).

TABLE 1

Genes induced by hypoxia in glioblastoma cell line, D247-MG

| | | | Fold | |
|---|---|---|---|---|
| SAGE Tag^{e} | Gene Symbol^{a} (Name) | Accession^{b} | Inc^{c} SAGE | Fold Inc^{c} PCR |
| TTTGTTAAAA | HOG18* (hypothetical protein FLJ20500) | NM_019058 | 10x | 5.7x |
| GCCACGTTGT | HOG3* (hypothetical protein DKFZp434K1210) | NM_017606 | 9x | 4.0x |
| GTGCTGGTGC | HFARP* or PGAR* (Ilepatic fibrinogen/angiopoietin- | NM_016109 | 8x | 12x |
| | related protein, PPAR-γ angiopoietin-related protein) | | | |
| CAGCCAAATA | HOG8* (similar to F-box only protein 6, a receptor | BC007832 | 8x | 2.1x |
| | for ubiquitination targets) | | | |
| CTTAAGAAAA | mig-6* (Mitogen-inducible gene 6) | AL137274 | 7x | 2.5x |
| TGTTAGAAAA | PLOD2 (Lysine hydroxylase 2) | NM_000935 | 5x | 4.4x |
| GATAGCACAG | IGFBP5 (Insulin-like growth factor binding protein 5) | L27560 | 4x | 4.6x |
| GCTCTCTATG | SSR4 (Translocon associated protein delta) | NM_006280 | 3x | 2.3x |

^{a}HUGO gene symbols are provided, or marked (*) if not yet available. Genes already known to be hypoxia are referenced.
^{b}GenBank or RefSeq accession number corresponding to the SAGE tag.
^{c}Fold increases (Fold Inc) are the ratio of hypoxic to normal transcript levels for SAGE and real-time PCR.
^{d}Not Tested.
^{e}SEQ ID NOS:23-30, respectively.

### Example 2

### Time Course of Gene Induction by Hypoxia

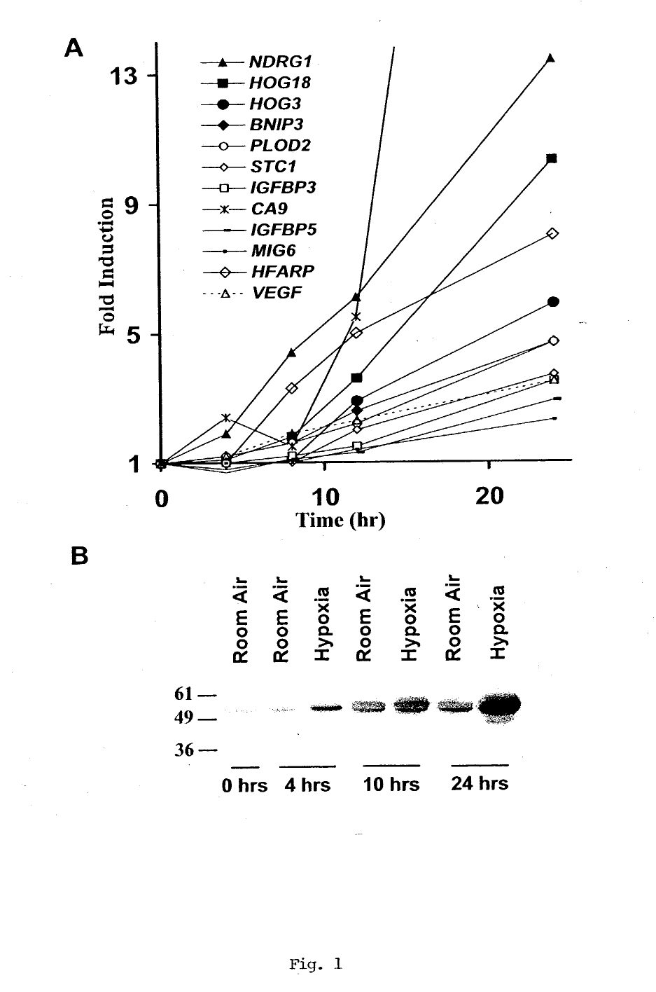

A time course of induction was performed on 12 hypoxia-inducible genes using real-time PCR (FIG. 1A). These genes all had a time course similar to VEGF, except for CA9, NDRG1, HFARP and HOG18, which were induced to a higher fold induction. Most of the genes required a 12-hour exposure prior to significant induction, implying an adaptation to chronic, rather than acute, hypoxia. Western blotting using an antibody to CA9 showed that protein levels increased with a time course similar to that of transcript levels (FIG. 1B).

### Example 3

### Regulation of Hypoxia-Induced Genes by HIF-1

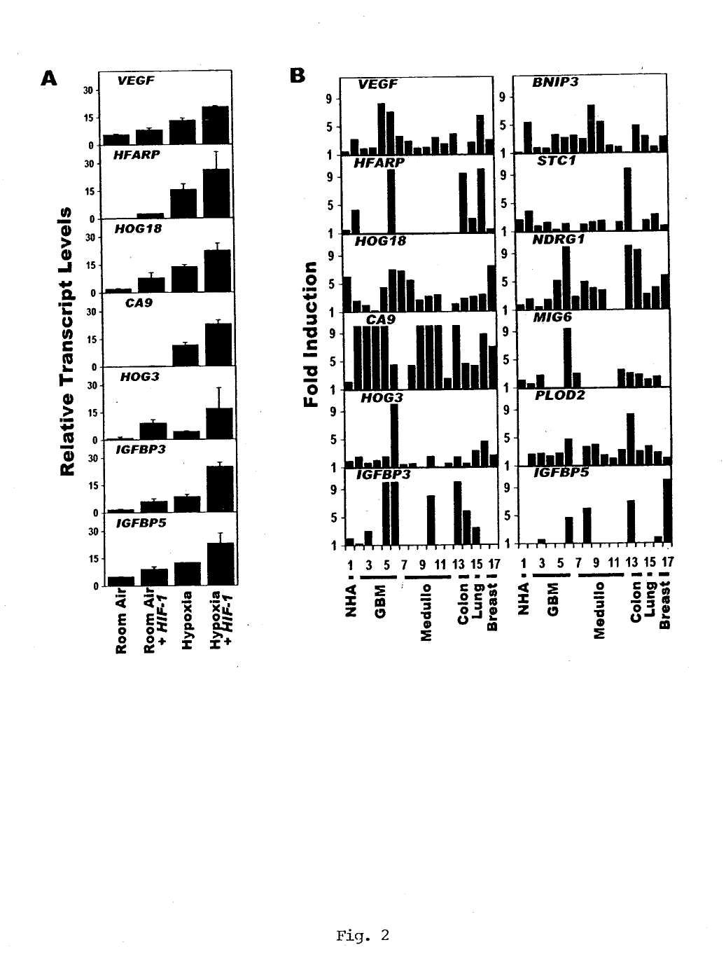

HOG18, HOG3, HFARP, CA9, IGFBP5 and IGFBP3 were tested to see if these genes might be regulated by HIF-1. VEGF, an HIF-1 regulated gene, was used as a positive control (Ravi R, et al., Genes Dev 14:34-44 (2000)). Standard transient transfection was able to insert HIF-1α subunit gene plasmid (or a lac-Z control plasmid) into about 20% of the D247-MG cells as demonstrated by β-galactosidase staining. All of the above genes showed a reproducible increase in expression due to HIF-1α at both atmospheric and 1% oxygen (FIG. 2A).

### Example 4

### Hypoxia-Induced Gene Expression in Malignant Cell Lines

HOG induction in malignant cell lines derived from commonly occurring cancers was determined by lowering the oxygen concentration from normal to 1.5% oxygen and measuring induction by real-time PCR. The 17 cell lines used were Normal Human Astrocytes (1), glioblastomas D263-MG (2), D392-MG (3), D502-MG (4), D566-MG (5) and U87 (6), medulloblastomas D283-Med (7), D341-Med (8), D425-Med (9), D556-Med (10), D581-Med (11) and UW228 (12), colon carcinomas SW480 (13) and HCT116 (14), non-small lung carcinomas NCI-H23 (15) and breast cancers SKBr3 (16) and MCF7 (17). Genes induced greater than 10-fold are displayed as 10-fold. The results are displayed in (FIG. 2B).

### Example 5

### In Vivo Studies of Hypoxia-Induced Genes

The in vivo response to hypoxic conditions in human solid tumors was examined. Pimonidazole, a bioreductive marker (Raleigh J A et al., Cancer Res 58:3765-8 (1998), was used to accurately mark the hypoxic cells (Wijffels K I, et al., Br J Cancer 83:674-83 (2000)) of cervical or head and neck tumors. Staining of adjacent frozen sections allowed determination of HOG expression co-localized with pimonidazole and other markers. Oropharynx carcinoma biopsies that were previously labeled with pimonidazole hydrochloride (Hypoxyprobe-1, Natural Pharmacia International Inc) and iododeoxyuridine (IdUrd), an S-phase marker, were obtained during diagnostic examination under anesthesia. Pimonidazole and IdUrd were injected intravenously, 2 h and 20 min, respectively, before biopsy as previously described (Wijffels K I et al., Br J Cancer 83:674-83 (2000)).

Immunohistochemical staining for CA9 was performed on 5-8 μm fresh frozen tissue sections using mouse monoclonal antibodies to the target hypoxia induced protein at a dilution of 3.2 mg/ml. The slides were fixed with acetone, blocked with horse serum, and sequentially incubated at room temperature with primary antibody, biotinylated secondary antibody, and avidin-biotin horseradish peroxidase complexes. Bound antibody was detected using 3,3′-diaminobenzidine and hydrogen peroxide, counterstained with 1% hematoxylin, and permanently mounted.

For visualization of pimonidazole and IdUrd, 5 μm sections were placed in pre-cooled acetone at 4° C. for 10 min, air-dried and rehydrated with PBS. Tissue DNA was denaturated in 2N HCl for 10 min. To neutralize pH, sections were rinsed in 0.1M Borax followed by rinsing in PBS. Sections were incubated for 45 min at 37° C. with 1 μg/ml anti-IdUrd and rabbit-anti-pimonidazole 1:200 in polyclonal liquid diluent. Next, sections were incubated for 90 min at room temperature in goat-anti-rabbit-ALEXAFLUOR488 (Molecular Probes, Eugene, Oreg.) and goat-anti-mouseCy3, both 1 μg/ml in polyclonal liquid diluent. Between incubations the sections were rinsed in PBS and finally mounted with Fluorostab.

Non-radioactive in situ hybridization was performed using digoxigenin-labeled antisense RNA probes. PCR was used to generate 350- to 600-bp products specific to each HOG and these products were subcloned into a pBluescript KS-(Stratagene). After growth in E. coli, the plasmid was cut at a unique poly-linker site to create a linear probe. Digoxigenin-labeled RNA probes, from both the sense and antisense strands, were generated using the digoxigenin RNA labeling reagents and either T7 or T3 polymerase (Roche Diagnostics). Alternatively, the T7 promoter was incorporated into an antisense primer and the RNA probes were generated as described earlier (St. Croix B, et al., Science 289:1197-202 (2000)). Fresh frozen sections are cut to 8 μm for in situ hybridization and processed as previously described (St. Croix B, et al., Science 289:1197-202 (2000)).

### Example 6

### Diagnosis and Localization of a Tumor in a Patient

Monoclonal antibodies to one of HOG3, HOG8, HOG18, HFARP, mig-6, PLOD2, SSR4, or IGFBP5 are coupled to ¹¹¹In via via N-succinimidyl-3-(tri-n-butylstanyl)benzoate (see, e.g., Zalutsky M and Narula A, Appl. Radiat. Isot. 38:1051 (1987). A pharmaceutical formulation of the labeled antibody is prepared in sterile pyrogen-free phosphate-buffered saline solution and administered intravenously to the subject. The tumor is localized using a gamma ray detector sensitive to ¹¹¹In emissions.

### Example 7

### Treatment of a Patient with a Brain Tumor

A subject found to have increased quantities of HOG3, HOG8, HOG18, HFARP, mig-6, PLOD2, SSR4, and IGFBP5 and having a solid tumor residing in the cerebral cortex is treated using therapeutic monoclonal antibodies that bind specifically to one of these proteins. The antibody is coupled to ¹³¹I via N-succinimidyl-3-(tri-n-butylstanyl)benzoate (see, e.g., Zalutsky M and Narula A, Appl. Radiat. Isot. 38:1051 (1987)) to form a therapeutic monoclonal antibody. The therapeutic monoclonal antibody is provided in a pharmaceutical formulation of sterile pyrogen-free phosphate-buffered saline solution and administered via intrathecal injection into the carotid artery. The patient receives 300 mCi of therapeutic antibody. The antibody is administered to the subject in a series of regular, periodic administrations.

We claim:

1. A method of inhibiting angiogenesis associated with wound healing, retinopathy, ischemia, inflammation, microvasculopathy, bone healing, skin inflammation, or follicular development, comprising:
providing to a subject in need thereof an antisense polynucleotide comprising 15 or more consecutive nucleotides of the complement of a sequence selected from the group consisting of SEQ ID NO:1 (HOG3), SEQ ID NO:3 (HOG8), SEQ ID NO:5 (HOG18), SEQ ID NO:9 (CA9), SEQ ID NO:11 (HXB), SEQ ID NO:13 (IGFBP5), SEQ ID NO:15 (HFARP), SEQ ID NO:17(STC1), SEQ ID NO:19 (mig-6) and SEQ ID NO:21 (SSR4), whereby angiogenesis is inhibited.

2. The method of claim 1 wherein the antisense polynucleotide is provided by administering an expression vector with expresses said antisense polynucleotide.

3. The method of claim 1 wherein the antisense polynucleotide is administered to the subject.

4. A method of inhibiting angiogenesis associated with wound healing, retinopathy, ischemia, inflammation, microvasculopathy, bone healing, skin inflammation, or follicular development, comprising:
administering to a subject in need thereof an antibody which specifically binds to a polypeptide selected from the group consisting of SEQ ID NO:2 (HOG3), SEQ ID NO:4 (HOG8), SEQ ID NO:6 (HOG18), SEQ ID NO:10 (CA9), SEQ ID NO:12 (HXB), SEQ ID NO:14 (IGFBP5), SEQ ID NO:16 (HFARP), SEQ ID NO:18 (STC1), SEQ ID NO:20 (mig-6) and SEQ ID NO:22 (SSR4), whereby angiogenesis is inhibited.

5. The method of claim 4 wherein the antibody is a human antibody.

6. The method of claim 4 wherein the antibody is a humanized antibody.

7. The method of claim 4 wherein the antibody is a chimeric antibody.

8. The method of claim 4 wherein the antibody is an antigen-binding fragment of an antibody.

9. The method of claim 8 wherein the antigen-binding fragment is a single-chain Fv fragment.

10. A method of promoting angiogenesis associated with wound healing, retinopathy, ischemia, inflammation, microvasculopathy, bone healing, skin inflammation, or follicular development, comprising:
administering to a subject in need thereof a polypeptide selected from the group consisting of SEQ ID NO:2 (HOG3), SEQ ID NO:4 (HOG8), SEQ ID NO:6 (HOG18), SEQ ID NO:10 (CA9), SEQ ID NO:12 (HXB), SEQ ID NO:14 (IGFBP5), SEQ-ID NO:16 (HFARP), SEQ ID NO:18 (STC™), SEQ ID NO:20 (mig-6) and SEQ ID NO:22 (SSR4), whereby angiogenesis is promoted.

11. A method of promoting angiogenesis associated with wound healing, retinopathy, ischemia, inflammation, microvasculopathy, bone healing, skin inflammation, or follicular development, comprising:
administering to a subject in need thereof a vector comprising a nucleotide sequence encoding a polypeptide selected from the group consisting of SEQ ID NO:2 (HOG3), SEQ ID NO:4 (HOG8), SEQ ID NO:6 (HOG18), SEQ ID NO:10 (CA9), SEQ ID NO:12 (HXB), SEQ ID NO:14 (IGFBP5), SEQ ID NO:16 (HFARP), SEQ ID NO:18 (STC1), SEQ ID NO:20 (mig-6) and SEQ ID NO:22 (SSR4) and a promotor, wherein the nucleotide sequence is operably linked to the promoter and is transcribed into a sense mRNA encoding said polypeptide upon transcription of the nucleotide sequence, whereby angiogenesis is promoted.

12. A method of treating a tumor, comprising:
providing to a subject in need thereof an antisense polynucleotide comprising 15 or more consecutive nucleotides of the complement of a sequence selected from the group consisting of SEQ ID NO:1 (HOG3), SEQ ID NO:3 (HOG8), SEQ ID NO:5 (HOG 18), SEQ ID NO:13 (IGFBP5), SEQ ID NO:15 (HFARP), SEQ ID NO:19 (mig-6) and SEQ ID NO:21 (SSR4), whereby the growth of the tumor is diminished.

13. The method of claim 12 wherein the antisense polynucleotide is provided by administering an expression vector which expresses said antisense polynucleotide.

14. The method of claim 12 wherein the antisense polynucleotide is administered to the subject.

15. A method of treating a tumor, comprising:
administering to a subject in need thereof an antibody which specifically binds to a polypeptide selected from the group consisting of SEQ ID NO:2 (HOG3), SEQ ID NO:4 (HOG8), SEQ ID NO:6 (HOG18), SEQ ID NO:14 (IGFBP5), SEQ ID NO:16 (HFARP), SEQ ID NO:20 (mig-6) and SEQ ID NO:22 (SSR4), whereby the growth of the tumor is diminished.

16. The method of claim 15 wherein the antibody is a human antibody.

17. The method of claim 15 wherein the antibody is a humanized antibody.

18. The method of claim 15 wherein the antibody is a chimeric antibody.

19. The method of claim 15 wherein the antibody is an antigen-binding fragment.

20. The method of claim 19 wherein the antigen-binding fragment is a single-chain Fv fragment.

21. The method of claim 15 wherein the antibody is covalently linked to a chemotherapeutic anti-tumor agent or a radiotherapeutic anti-tumor agent.

22. A method of diagnosing cancer in a subject, comprising:
quantifying a polypeptide selected from the group consisting of SEQ ID NO:2 (HOG3), SEQ ID NO:4 (HOG8), SEQ ID NO:6 (HOG18), SEQ ID NO:8 (PLOD2), SEQ ID NO:14 (IGFBP5), SEQ ID NO:16 (HFARP), SEQ ID NO:20 (mig-6) and SEQ ID NO:22 (SSR4), in a test sample suspected of being neoplastic from the subject and in a non-neoplastic control sample;
comparing the quantity of the polypeptide in a test sample suspected of being neoplastic with the quantity of the polypeptide in a non-neoplastic control sample; and
identifying the test sample as cancerous if the quantity of the polypeptide is higher in the test sample than in the control sample.

23. The method of claim 22 wherein the cancer is selected from the group consisting of breast cancer, colon cancer, and lung cancer.

24. The method of claim 22 wherein the cancer is glioblastoma.

25. The method of claim 22 wherein the step of quantifying is performed using an immunoassay.

26. The method of claim 25 wherein the step of quantifying is performed using Western blot or immunohistochemical assay.

27. A method of diagnosing cancer in a subject, comprising:
quantifying an mRNA selected from the group consisting of SEQ ID NO:1 (HOG3), SEQ ID NO:3 (HOG8), SEQ ID NO:5 (HOG18), SEQ ID NO:7 (PLOD2), SEQ ID NO:13 (IGFBP5), SEQ ID NO:15 (HFARP), SEQ ID NO:19 (mig-6) and SEQ ID NO:21 (SSR4), in a test sample suspected of being neoplastic from the subject and in a non-neoplastic control sample;
comparing the quantity of the mRNA in a test sample suspected of being neoplastic with the quantity of the mRNA in a non-neoplastic control sample; and
identifying the test sample as cancerous if the quantity of the mRNA is higher in the test sample than in the control sample.

28. The method of claim 27 wherein the step of quantifying employs a nucleic acid hybridization to a probe.

29. The method of claim 28 wherein the step of quantifying is performed using a Northern blot.

30. The method of claim 28 wherein the step of quantifying is performed using hybridization to probes in an array.

31. The method of claim 27 wherein mRNA is amplified before quantification.

32. The method of claim 27 wherein the step of quantifying is performed using RT-PCR.

33. A method of imaging a tumor comprising:
administering to a subject or to a tissue sample from a subject an antibody which specifically binds to a polypeptide selected from the group consisting of SEQ ID NO:2 (HOG3), SEQ ID NO:4 (HOG8), SEQ ID NO:6 (HOG18), SEQ ID NO:8 (PLOD2), SEQ ID NO:14 (IGFBP5), SEQ ID NO:16 (HFARP), SEQ ID NO:20 (mig-6) and SEQ ID NO:22 (SSR4), wherein the antibody is covalently linked to a label; and
detecting the label, whereby an image is formed of the distribution of the label in the subject or tissue sample.

34. The method of claim 33 wherein the label is radioactive, fluorescent, or colored.


SEQUENCE LISTING (30 sequences)

SEQ ID NO: 1 - DNA, 2133 bp, Homo sapiens
agggatttccccatttctgtttgctgcctgaaagcaggatgaggaaggccaaggagagtccttgcacccgtgagcgtcaggatgaggaaatgacaggaggaagacgtgggtttgggttagtggctgctggcgttttggcccttggtgtttctggagcctccagggatctaggggagcctgggctgcgtgcatgtcgataagcagagctgttcttggggagaaggagggaggtctcgggagtgtagcaccatgccaaccagccctgcgcgaagacagagtgagccacgcccggatggcagggcatgtttctgttttggtgtctcactttcctcccagcgtgacttatttggggattcctcagggcctactggaatgtgactgcccactgcccagctgcctcgggtacaagtcctggccctatgtcccagctgtcaggggctcagggaatcctacccagccacctgtcctgggatggagtgtcagcatccaccccttggttgtcatcgaggccgccctcccagtcctgggtgaagatatttgggccaccagggctcccttggccccttcacgtaggaaatagacacgtgctttttaatgcaggacactttgagtgttacaaaatctgtagacctggcagtagggtcatgatgttgggaagggtgtagtgccctaggttggtgacagaagggacagacacttgtgcacaggtgtctttggtgatggggtttttttttttataacttagtaaaaaaaaaaaaaaatgtatgtggaattctgtctcttggtaaagctcaaagccaggctagcctgaggtggcgcagggctctccttcctgtcccttcgatctccttgagaattaagagctggcagctgctgatggtgtttcccaacccccctcacttcccaagacaacccccagcttcaggtcctcatggggaggggagggcacgttcttgacacatgggaacttcgctcaggagggcctccccttcccctctccctcagagttttcactgccgtctcgtctttagaaagctgtttgaattccccccgcccccagtttggaccgtgtagatataactggatatacggatttttctctttgtgcaggcttcttatgccgttggtatacagggcaggaaagagaggaataaagggagagagcagtgtggaaaccacggtggttttgctttgttcttactaggttttggtgccaccttccctgcctgcgcttgtgccccctctcctccttggcactggcggcctccttgcctcccttccacccgtgctgccatcccgtgcctgtcgtgttggttcttcacacgtgctctgttctcggggttgttccattcatgccttcttggagggtgagggtggcttgggaaccgacccagtgatcatgcctactttcttctttgtatctccctccttcccagcccacccgggcagcagactctgatggaaggaaggtgccgtaggtgggcttttagaaactaacgggactggttttcaaagcagttatcttgggaaactgtttattccagcgatgtgacttttttcagaatatttcttggaatcatattcagagtctggggctgtgtgttgagcagccttaaggatgctagacactcatttagtgcccagggagtccagcgaatgacgtctgtggccaagcgaggtctcaggtgcaaagcaaaaggaccatttaaagtaaaatagcttggattcaatcatgtgacttttaaattggctcagaaagcaattttgtaatttcagagagtgttttgagccatggccacgttgtcattgtgagtctatagcttgactccttggagaacaatattcatttggttgtggagactgatttgctgggagaaatctgtcctgttactttctggtcatcccaggttctgacttttaccaggggcaaaaaaaaaaaaaaaaagcaagagggagataaatcccatctgtgagtttgtcttattggcgcctttttcctcagctgtcttccaagtattatttttactgttaaaaaattttttaaaaatgtgaaatgtaatgtttttacagcaacaatatgaaatatattttataaggaataaaatggtaccttgtctgaaaaaaaaaaaaaaa

SEQ ID NO: 2 - protein, 129 aa, Homo sapiens
MSISRAVLGEKEGGLGSVAPCQPALREDRVSHARMAGHVSVLVSHFPPSVTYLGIPQGLLECDCPLPSCLGYKSWPYVPAVRGSGNPTQPPVLGWSVSIHPLVVIEAALPVLGEDIWATRAPLAPSRRK

SEQ ID NO: 3 - DNA, 1768 bp, Homo sapiens
ggcacgagggcttgggcggcccagcggatcgtgccgcggcggccgagcgcagctacaggagggtgtccagaagccacaagccatggctgtggggaacatcaacgagctgcccgagaacatcctgctggagctgttcacgcacgtgcccgcccgccagctgctgctgaactgccgcctggtctgcagcctctggcgggacctcatcgacctcgtgaccctctggaaacgcaagtgcctgcgagagggcttcatcactgaggactgggaccagcccgtggccgactggaagatcttctacttcttacggagcctgcacaggaacctcctgcacaacccgtgcgctgaagaggggttcgagttctggagcctggatgtgaatggaggcgatgagtggaaggtggaggatctctctcgagaccagaggaaggaattccccaatgaccaggttcgcagccaggccagattgcgggtccaagtaccagctgtgcgttcagctcctgtcgtccgcgcacgcgcctctggggaccttccagccagacccggcgaccatccagcagaagagcgatgccaagtggagggaggtctcccacacattctccaactacccgcccggcgtccgctacatctggtttcagcacggcggcgtggacactcattactgggccggctggtacggcccgagggtcaccaacagcagcatcaccatcgggcccccgctgccctgacaccccctgagcccccatctgctgaaccctgactggtaaacaactgctgtcagaaaagggctgggcttgggaaggggaggtggaggccaggtgtccccagacctctaacccttgcccctagcagcctcttctttgtggagcctctcagtgtgggcagccctcgcatgctggggtcgggccagctctccccgaaaggtcttgacctgaatgatggccggggaagcctgcgtgtgcccctttcagagacggagcacctgagatgtgggaggtgcagcatgttcccctgggcccctcagaaagtcgagcttggaggccagcctggatctgtctctcccttcccctcctgggaccattctacctgtgttctttgaccctcggagcagggacaggcaagacaactggcaagcttgcagctgccctgatggtgcaggtgcagggaggtgaccatgtaactctgaccaatctgggaagtggagggtgggctcatgggccgtgccctgcccctgtctgctgctcccagtctttcgctctgcctgcctgctcagaagaggtggctttggcccagaggctcaggccgggactgagatggacagacccagggtggggtggggtccaggtcgggtgtggactgtcctcactgtcagtggagccccagaagctagatgggtaccaggtggggttaggttcccagaggactgagggaatcctgtacaggatgtcccagggtagatggggagcaggattgggacctgctctgacagctggacacatgagccctggatgagtatggtagggggtttgaagaatcccctgtccacctcccaaatccaggcccggccccctctggcttggagagcattccaagcccccaccccacccctagaactgccattcccaagacctctgtctcccagccaaccacccttggaacttgcctcttgtcctgctggaaagatagcagtgttctcctgacttcgccctactgcatgcagccaaataaaaggtgtgcccagtctaaaaaaaaaaaaaaaaaaaaaaaaaaa

SEQ ID NO: 4 - protein, 224 aa, Homo sapiens
MAVGNINELPENILLELFTHVPARQLLLNCRLVCSLWRDLIDLVTLWKRKCLREGFITEDWDQPVADWKIFYFLRSLHRNLLHNPCAEEGFEFWSLDVNGGDEWKVEDLSRDQRKEFPNDQVRSQARLRVQVPAVRSAPVVRARASGDLPARPGDHPAEERCQVEGGLPHILQLPARRPLHLVSARRRGHSLLGRLVRPEGHQQQHHHRAPAALTPPEPPSAEP

SEQ ID NO: 5 - DNA, 1760 bp, Homo sapiens
gcagcaggccaagggggaggtgcgagcgtggacctgggacgggtctgggcggctctcggtggttggcacgggttcgcacacccattcaagcggcaggacgcacttgtcttagcagttctcgctgaccgcgctagctgcggcttctacgctccggcactctgagttcatcagcaaacgccctggcgtctgtcctcaccatgcctagcctttgggaccgcttctcgtcgtcgtccacctcctcttcgccctcgtccttgccccgaactcccaccccagatcggccgccgcgctcagcctgggggtcggcgacccgggaggaggggtttgaccgctccacgagcctggagagctcggactgcgagtccctggacagcagcaacagtggcttcgggccggaggaagacacggcttacctggatggggtgtcgttgcccgacttcgagctgctcagtgaccctgaggatgaacacttgtgtgccaacctgatgcagctgctgcaggagagcctggcccaggcgcggctgggctctcgacgccctgcgcgcctgctgatgcctagccagttggtaagccaggtgggcaaagaactactgcgcctggcctacagcgagccgtgcggcctgcggggggcgctgctggacgtctgcgtggagcagggcaagagctgccacagcgtgggccagctggcactcgaccccagcctggtgcccaccttccagctgaccctcgtgctgcgcctggactcacgactctggcccaagatccaggggctgtttagctccgccaactctcccttcctccctggcttcagccagtccctgacgctgagcactggcttccgagtcatcaagaagaagctgtacagctcggaacagctgctcattgaggagtgttgaacttcaacctgagggggccgacagtgccctccaagacagagacgactgaacttttggggtggagactagaggcaggagctgagggactgattccagtggttggaaaactgaggcagccacctaaggtggaggtgggggaatagtgtttcccaggaagctcattgagttgtgtgcgggtggctgtgcattggggacacatacccctcagtactgtagcatggaacaaaggcttaggggccaacaaggcttccagctggatgtgtgtgtagcatgtaccttattatttttgttactgacagttaacagtggtgtgacatccagagagcagctgggctgctcccgccccagcctggcccagggtgaaggaagaggcacgtgctcctcagagcagccggagggaggggggaggtcggaggtcgtggaggtggtttgtgtatcttactggtctgaagggaccaagtgtgtttgttgtttgttttgtatcttgtttttctgatcggagcatcactactgacctgttgtaggcagctatcttacagacgcatgaatgtaagagtaggaaggggtgggtgtcagggatcacttgggatctttgacacttgaaaaattacacctggcagctgcgtttaagccttcccccatcgtgtactgcagagttgagctggcaggggaggggctgagagggtgggggctggaacccctccccgggaggagtgccatctgggtcttccatctagaactgtttacatgaagataagatactcactgttcatgaatacacttgatgttcaagtattaagacctatgcaatattttttacttttctaataaacatgtttgttaaaacaaaaaaaaaaaaaaaaa

SEQ ID NO: 6 - protein, 232 aa, Homo sapiens
MPSLWDRFSSSSTSSSPSSLPRTPTPDRPPRSAWGSATREEGFDRSTSLESSDCESLDSSNSGFGPEEDTAYLDGVSLPDFELLSDPEDEHLCANLMQLLQESLAQARLGSRRPARLLMPSQLVSQVGKELLRLAYSEPCGLRGALLDVCVEQGKSCHSVGQLALDPSLVPTFQLTLVLRLDSRLWPKIQGLFSSANSPFLPGFSQSLTLSTGFRVIKKKLYSSEQLLIEEC

SEQ ID NO: 7 - DNA, 3503 bp, Homo sapiens
atggggggatgcacggtgaagcctcagctgctgctcctggcgctcgtcctccacccctggaatccctgtctgggtgcggactcggagaagccctcgagcatccccacagataaattattagtcataactgtagcaacaaaagaaagtgatggattccatcgatttatgcagtcagccaaatatttcaattatactgtgaaggtccttggtcaaggagaagaatggagaggtggtgatggaattaatagtattggagggggccagaaagtgagattaatgaaagaagtcatggaacactatgctgatcaagatgatctggttgtcatgtttactgaatgctttgatgtcatatttgctggtggtccagaagaagttctaaaaaaattccaaaaggcaaaccacaaagtggtctttgcagcagatggaattttgtggccagataaaagactagcagacaagtatcctgttgtgcacattgggaaacgctatctgaattcaggaggatttattggctatgctccatatgtcaaccgtatagttcaacaatggaatctccaggataatgatgatgatcagctcttttacactaaagtttacattgatccactgaaaagggaagctattaacatcacattggatcacaaatgcaaaattttccagaccttaaatggagctgtagatgaagttgttttaaaatttgaaaatggcaaagccagagctaagaatacattttatgaaacattaccagtggcaattaatggaaatggacccaccaagattctcctgaattattttggaaactatgtacccaattcatggacacaggataatggctgcactctttgtgaattcgatacagtcgacttgtctgcagtagatgtccatccaaacgtatcaataggtgtttttattgagcaaccaaccccttttctacctcggtttctggacatattgttgacactggattacccaaaagaagcacttaaactttttattcataacaaagaagtttatcatgaaaaggacatcaaggtattttttgataaagctaagcatgaaatcaaaactataaaaatagtaggaccagaagaaaatctaagtcaagcggaagccagaaacatgggaatggacttttgccgtcaggatgaaaagtgtgattattactttagtgtggatgcagatgttgttttgacaaatccaaggactttaaaaattttgattgaacaaaacagaaagatcattgctcctcttgtaactcgtcatggaaagctgtggtccaatttctggggagcattgagtcctgatggatactatgcacgatctgaagattatgtggatattgttcaagggaatagagtaggagtatggaatgtcccatatatggctaatgtgtacttaattaaaggaaagacactccgatcagagatgaatgaaaggaactattttgttcgtgataaactggatcctgatatggctctttgccgaaatgctagagaaatgggtgtatttatgtacatttctaatagacatgaatttggaaggctattatccactgctaattacaatacttcccattataacaatgacctctggcagatttttgaaaatcctgtggactggaaggaaaagtatataaaccgtgattattcaaagattttcactgaaaatatagttgaacagccctgtccagatgtcttttggttccccatattttctgaaaaagcctgtgatgaattggtagaagaaatggaacattacggcaaatggtctgggggaaaacatcatgatagccgtatatctggtggttatgaaaatgtcccaactgatgatatccacatgaagcaagttgatctggagaatgtatggcttgattttatccgggagttcattgcaccagttacactgaaggtctttgcaggctattatacgaagggatttgcactactgaattttgtagtaaaatactcccctgaacgacagcgttctcttcgtcctcatcatgatgcttctacatttaccataaacattgcacttaataacgtgggagaagactttcagggaggtggttgcaaatttctaaggtacaattgctctattgagtcaccacgaaaaggctggagcttcatgcatcctgggagactcacacatttgcatgaaggacttcctgttaaaaatggaacaagatacattgcagtgtcatttatagatccctaagttatttacttttcattgaattgaaatttattttgggtgaatgactggcatgaacacgtctttgaagttgtggctgagaagatgagaggaatatttaaataacatcaacagaacaacttcactttgggccaaacatttgaaaaactttttataaaaaattgtttgatatttcttaatgtctgctctgagccttaaaacacagattgaagaagaaaagaaagaaaaaacttaaatatttatttctatgctttgttgcctctgagaataatgacaatttatgaatttgtgtttcaaattgataaaatatttaggtacaaataacaagactaataatattttcttatttaaaaaaagcatgggaagatttttatttatcaaaatatagaggaaatgtagacaaaatggatataaatgaaaattaccatgttgtaaaaccttgaaaatcagattctaactgattgtatgcaactaagtatttctgaacacctatgcaggtcttatttacagtgttactaagggaacacacaaagaattacacaacgttttcctcaagaaaatggtacaaaacacaaccgaggagcgtatacagttgaaaacatttttgttttgattggaaggcagattattttatattagtattaaaaatcaaaccctatgtttctttcagatgaatcttccaaagtggattatattaagcaggtattagatttagaaaacctttccatttcttaaagtattatcaagtgtcaagatcagcaagtgtccttaagtcaaataggtttttttttgttggtggttgtgcttgctttccttttttagaaagttctagaaaataggaaaacgaaaaatttcattgagatgagtagtgcatttaattattttttaaaaaactttttaagtacttgaattttatatcaggaaaacaaagttgttgagccttgcttcttccgttttgccctttgtctcgctccttattcttttttggggggagggttatttgcttttttatcttcctggcataatttccattttattcttctgagtgtctatgttaacttccctctatcccgcttataaaaaaattctccaacaaaaatacttgttgacttgatgttttatcacttctctaagtaaggttgaaatatccttattgtagctactgtttttaatgtaaaggttaaacttgaaaagaaattcttaatcacggtgccaaaattcattttctaacaccatgtgttagaaaattataaaaaataaaataattttaaaaaaaaaaaaaaaaaaaaaaa

SEQ ID NO: 8 - protein, 737 aa, Homo sapiens
MGGCTVKPQLLLLALVLHPWNPCLGADSEKPSSIPTDKLLVITVATKESDGFHRFMQSAKYFNYTVKVLGQGEEWRGGDGINSIGGGQKVRLMKEVMEHYADQDDLVVMFTECFDVIFAGGPEEVLKKFQKANHKVVFAADGILWPDKRLADKYPVVHIGKRYLNSGGFIGYAPYVNRIVQQWNLQDNDDDQLFYTKVYIDPLKREAINITLDHKCKIFQTLNGAVDEVVLKFENGKARAKNTFYETLPVAINGNGPTKILLNYFGNYVPNSWTQDNGCTLCEFDTVDLSAVDVHPNVSIGVFIEQPTPFLPRFLDILLTLDYPKEALKLFIHNKEVYHEKDIKVFFDKAKHEIKTIKIVGPEENLSQAEARNMGMDFCRQDEKCDYYFSVDADVVLTNPRTLKILIEQNRKIIAPLVTRHGKLWSNFWGALSPDGYYARSEDYVDIVQGNRVGVWNVPYMANVYLIKGKTLRSEMNERNYFVRDKLDPDMALCRNAREMGVFMYISNRHEFGRLLSTANYNTSHYNNDLWQIFENPVDWKEKYINRDYSKIFTENIVEQPCPDVFWFPIFSEKACDELVEEMEHYGKWSGGKHHDSRISGGYENVPTDDIHMKQVDLENVWLDFIREFIAPVTLKVFAGYYTKGFALLNFVVKYSPERQRSLRPHHDASTFTINIALNNVGEDFQGGGCKFLRYNCSIESPRKGWSFMHPGRLTHLHEGLPVKNGTRYIAVSFIDP

SEQ ID NO: 9 - DNA, 1552 bp, Homo sapiens
gcccgtacacaccgtgtgctgggacaccccacagtcagccgcatggctcccctgtgccccagcccctggctccctctgttgatcccggcccctgctccaggcctcactgtgcaactgctgctgtcactgctgcttctgatgcctgtccatccccagaggttgccccggatgcaggaggattcccccttgggaggaggctcttctggggaagatgacccactgggcgaggaggatctgcccagtgaagaggattcacccagagaggaggatccacccggagaggaggatctacctggagaggaggatctacctggagaggaggatctacctgaagttaagcctaaatcagaagaagagggctccctgaagttagaggatctacctactgttgaggctcctggagatcctcaagaaccccagaataatgcccacagggacaaagaaggggatgaccagagtcattggcgctatggaggcgacccgccctggccccgggtgtccccagcctgcgcgggccgcttccagtccccggtggatatccgcccccagctcgccgccttctgcccggccctgcgccccctggaactcctgggcttccagctcccgccgctcccagaactgcgcctgcgcaacaatggccacagtgtgcaactgaccctgcctcctgggctagagatggctctgggtcccgggcgggagtaccgggctctgcagctgcatctgcactggggggctgcaggtcgtccgggctcggagcacactgtggaaggccaccgtttccctgccgagatccacgtggttcacctcagcaccgcctttgccagagttgacgaggccttggggcgcccgggaggcctggccgtgttggccgcctttctggaggagggcccggaagaaaacagtgcctatgagcagttgctgtctcgcttggaagaaatcgctgaggaaggctcagagactcaggtcccaggactggacatatctgcactcctgccctctgacttcagccgctacttccaatatgaggggtctctgactacaccgccctgtgcccagggtgtcatctggactgtgtttaaccagacagtgatgctgagtgctaagcagctccacaccctctctgacaccctgtggggacctggtgactctcggctacagctgaacttccgagcgacgcagcctttgaatgggcgagtgattgaggcctccttccctgctggagtggacagcagtcctcgggctgctgagccagtccagctgaattcctgcctggctgctggtgacatcctagccctggtttttggcctcctttttgctgtcaccagcgtcgcgttccttgtgcagatgagaaggcagcacagaaggggaaccaaagggggtgtgagctaccgcccagcagaggtagccgagactggagcctagaggctggatcttggagaatgtgagaagccagccagaggcatctgagggggagccggtaactgtcctgtcctgctcattatgccacttccttttaactgccaagaaattttttaaaataaatatttataat

SEQ ID NO: 10 - protein, 459 aa, Homo sapiens
MAPLCPSPWLPLLIPAPAPGLTVQLLLSLLLLMPVHPQRLPRMQEDSPLGGGSSGEDDPLGEEDLPSEEDSPREEDPPGEEDLPGEEDLPGEEDLPEVKPKSEEEGSLKLEDLPTVEAPGDPQEPQNNAHRDKEGDDQSHWRYGGDPPWPRVSPACAGRFQSPVDIRPQLAAFCPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQLHTLSDTLWGPGDSRLQLNFRATQPLNGRVIEASFPAGVDSSPRAAEPVQLNSCLAAGDILALVFGLLFAVTSVAFLVQMRRQHRRGTKGGVSYRPAEVAETGA

SEQ ID NO: 11 - DNA, 7560 bp, Homo sapiens
accggccacagcctgcctactgtcacccgcctctcccgcgcgcagatacacgcccccgcctccgtgggcacaaaggcagcgctgctggggaactcgggggaacgcgcacgtgggaaccgccgcagctccacactccaggtacttcttccaaggacctaggtctctcgcccatcggaaagaaaataattctttcaagaagatcagggacaactgatttgaagtctactctgtgcttctaaatccccaattctgctgaaagtgaatccctagagccctagagccccagcagcacccagccaaacccacctccaccatgggggccatgactcagctgttggcaggtgtctttcttgctttccttgccctcgctaccgaaggtggggtcctcaagaaagtcatccggcacaagcgacagagtggggtgaacgccaccctgccagaagagaaccagccagtggtgtttaaccacgtttacaacatcaagctgccagtgggatcccagtgttcggtggatctggagtcagccagtggggagaaagacctggcaccgccttcagagcccagcgaaagctttcaggagcacacagtagatggggaaaaccagattgtcttcacacatcgcatcaacatcccccgccgggcctgtggctgtgccgcagcccctgatgttaaggagctgctgagcagactggaggagctggagaacctggtgtcttccctgagggagcaatgtactgcaggagcaggctgctgtctccagcctgccacaggccgcttggacaccaggcccttctgtagcggtcggggcaacttcagcactgaaggatgtggctgtgtctgcgaacctggctggaaaggccccaactgctctgagcccgaatgtccaggcaactgtcaccttcgaggccggtgcattgatgggcagtgcatctgtgacgacggcttcacgggcgaggactgcagccagctggcttgccccagcgactgcaatgaccagggcaagtgcgtgaatggagtctgcatctgtttcgaaggctacgccggggctgactgcagccgtgaaatctgcccagtgccctgcagtgaggagcacggcacatgtgtagatggcttgtgtgtgtgccacgatggctttgcaggcgatgactgcaacaagcctctgtgtctcaacaattgctacaaccgtggacgatgcgtggagaatgagtgcgtgtgtgatgagggtttcacgggcgaagactgcagtgagctcatctgccccaatgactgcttcgaccggggccgctgcatcaatggcacctgctactgcgaagaaggcttcacaggtgaagactgcgggaaacccacctgcccacatgcctgccacacccagggccggtgtgaggaggggcagtgtgtatgtgatgagggctttgccggtttggactgcagcgagaagaggtgtcctgctgactgtcacaatcgtggccgctgtgtagacgggcggtgtgagtgtgatgatggtttcactggagctgactgtggggagctcaagtgtcccaatggctgcagtggccatggccgctgtgtcaatgggcagtgtgtgtgtgatgagggctatactggggaggactgcagccagctacggtgccccaatgactgtcacagtcggggccgctgtgtcgagggcaaatgtgtatgtgagcaaggcttcaagggctatgactgcagtgacatgagctgccctaatgactgtcaccagcacggccgctgtgtgaatggcatgtgtgtttgtgatgacggctacacaggggaagactgccgggatcgccaatgccccagggactgcagcaacaggggcctctgtgtggacggacagtgcgtctgtgaggacggcttcaccggccctgactgtgcagaactctcctgtccaaatgactgccatggccagggtcgctgtgtgaatgggcagtgcgtgtgccatgaaggatttatgggcaaagactgcaaggagcaaagatgtcccagtgactgtcatggccagggccgctgcgtggacggccagtgcatctgccacgagggcttcacaggcctggactgtggccagcactcctgccccagtgactgcaacaacttaggacaatgcgtctcgggccgctgcatctgcaacgagggctacagcggagaagactgctcagaggtgtctcctcccaaagacctcgttgtgacagaagtgacggaagagacggtcaacctggcctgggacaatgagatgcgggtcacagagtaccttgtcgtgtacacgcccacccacgagggtggtctggaaatgcagttccgtgtgcctggggaccagacgtccaccatcatccaggagctggagcctggtgtggagtactttatccgtgtatttgccatcctggagaacaagaagagcattcctgtcagcgccagggtggccacgtacttacctgcacctgaaggcctgaaattcaagtccatcaaggagacatctgtggaagtggagtgggatcctctagacattgcttttgaaacctgggagatcatcttccggaatatgaataaagaagatgagggagagatcaccaaaagcctgaggaggccagagacctcttaccggcaaactggtctagctcctgggcaagagtatgagatatctctgcacatagtgaaaaacaatacccggggccctggcctgaagagggtgaccaccacacgcttggatgcccccagccagatcgaggtgaaagatgtcacagacaccactgccttgatcacctggttcaagcccctggctgagatcgatggcattgagctgacctacggcatcaaagacgtgccaggagaccgtaccaccatcgatctcacagaggacgagaaccagtactccatcgggaacctgaagcctgacactgagtacgaggtgtccctcatctcccgcagaggtgacatgtcaagcaacccagccaaagagaccttcacaacaggcctcgatgctcccaggaatcttcgacgtgtttcccagacagataacagcatcaccctggaatggaggaatggcaaggcagctattgacagttacagaattaagtatgcccccatctctggaggggaccacgctgaggttgatgttccaaagagccaacaagccacaaccaaaaccacactcacaggtctgaggccgggaactgaatatgggattggagtttctgctgtgaaggaagacaaggagagcaatccagcgaccatcaacgcagccacagagttggacacgcccaaggaccttcaggtttctgaaactgcagagaccagcctgaccctgctctggaagacaccgttggccaaatttgaccgctaccgcctcaattacagtctccccacaggccagtgggtgggagtgcagcttccaagaaacaccacttcctatgtcctgagaggcctggaaccaggacaggagtacaatgtcctcctgacagccgagaaaggcagacacaagagcaagcccgcacgtgtgaaggcatccactgaacaagcccctgagctggaaaacctcaccgtgactgaggttggctgggatggcctcagactcaactggaccgcggctgaccaggcctatgagcactttatcattcaggtgcaggaggccaacaaggtggaggcagctcggaacctcaccgtgcctggcagccttcgggctgtggacataccgggcctcaaggctgctacgccttatacagtctccatctatggggtgatccagggctatagaacaccagtgctctctgctgaggcctccacaggggaaactcccaatttgggagaggtcgtggtggccgaggtgggctgggatgccctcaaactcaactggactgctccagaaggggcctatgagtactttttcattcaggtgcaggaggctgacacagtagaggcagcccagaacctcaccgtcccaggaggactgaggtccacagacctgcctgggctcaaagcagccactcattataccatcaccatccgcggggtcactcaggacttcagcacaacccctctctctgttgaagtcttgacagaggaggttccagatatgggaaacctcacagtgaccgaggttagctgggatgctctcagactgaactggaccacgccagatggaacctatgaccagtttactattcaggtccaggaggctgaccaggtggaagaggctcacaatctcacggttcctggcagcctgcgttccatggaaatcccaggcctcagggctggcactccttacacagtcaccctgcacggcgaggtcaggggccacagcactcgaccccttgctgtagaggtcgtcacagaggatctcccacagctgggagatttagccgtgtctgaggttggctgggatggcctcagactcaactggaccgcagctgacaatgcctatgagcactttgtcattcaggtgcaggaggtcaacaaagtggaggcagcccagaacctcacgttgcctggcagcctcagggctgtggacatcccgggcctcgaggctgccacgccttatagagtctccatctatggggtgatccggggctatagaacaccagtactctctgctgaggcctccacagccaaagaacctgaaattggaaacttaaatgtttctgacataactcccgagagcttcaatctctcctggatggctaccgatgggatcttcgagacctttaccattgaaattattgattccaataggttgctggagactgtggaatataatatctctggtgctgaacgaactgcccatatctcagggctaccccctagtactgattttattgtctacctctctggacttgctcccagcatccggaccaaaaccatcagtgccacagccacgacagaggccctgccccttctggaaaacctaaccatttccgacattaatccctacgggttcacagtttcctggatggcatcggagaatgcctttgacagctttctagtaacggtggtggattctgggaagctgctggacccccaggaattcacactttcaggaacccagaggaagctggagcttagaggcctcataactggcattggctatgaggttatggtctctggcttcacccaagggcatcaaaccaagcccttgagggctgagattgttacagaagccgaaccggaagttgacaaccttctggtttcagatgccaccccagacggtttccgtctgtcctggacagctgatgaaggggtcttcgacaattttgttctcaaaatcagagataccaaaaagcagtctgagccactggaaataaccctacttgcccccgaacgtaccagggacttaacaggtctcagagaggctactgaatacgaaattgaactctatggaataagcaaaggaaggcgatcccagacagtcagtgctatagcaacaacagccatgggctccccaaaggaagtcattttctcagacatcactgaaaattcggctactgtcagctggagggcacccacggcccaagtggagagcttccggattacctatgtgcccattacaggaggtacaccctccatggtaactgtggacggaaccaagactcagaccaggctggtgaaactcatacctggcgtggagtaccttgtcagcatcatcgccatgaagggctttgaggaaagtgaacctgtctcagggtcattcaccacagctctggatggcccatctggcctggtgacagccaacatcactgactcagaagccttggccaggtggcagccagccattgccactgtggacagttatgtcatctcctacacaggcgagaaagtgccagaaattacacgcacggtgtccgggaacacagtggagtatgctctgaccgacctcgagcctgccacggaatacacactgagaatctttgcagagaaagggccccagaagagctcaaccatcactgccaagttcacaacagacctcgattctccaagagacttgactgctactgaggttcagtcggaaactgccctccttacctggcgacccccccgggcatcagtcaccggttacctgctggtctatgaatcagtggatggcacagtcaaggaagtcattgtgggtccagataccacctcctacagcctggcagacctgagcccatccacccactacacagccaagatccaggcactcaatgggcccctgaggagcaatatgatccagaccatcttcaccacaattggactcctgtaccccttccccaaggactgctcccaagcaatgctgaatggagacacgacctctggcctctacaccatttatctgaatggtgataaggctcaggcgctggaagtcttctgtgacatgacctctgatgggggtggatggattgtgttcctgagacgcaaaaacggacgcgagaacttctaccaaaactggaaggcatatgctgctggatttggggaccgcagagaagaattctggcttgggctggacaacctgaacaaaatcacagcccaggggcagtacgagctccgggtggacctgcgggaccatggggagacagcctttgctgtctatgacaagttcagcgtgggagatgccaagactcgctacaagctgaaggtggaggggtacagtgggacagcaggtgactccatggcctaccacaatggcagatccttctccacctttgacaaggacacagattcagccatcaccaactgtgctctgtcctacaaaggggctttctggtacaggaactgtcaccgtgtcaacctgatggggagatatggggacaataaccacagtcagggcgttaactggttccactggaagggccacgaacactcaatccagtttgctgagatgaagctgagaccaagcaacttcagaaatcttgaaggcaggcgcaaacgggcataaattggagggaccactgggtgagagaggaataaggcggcccagagcgaggaaaggattttaccaaagcatcaatacaaccagcccaaccatcggtccacacctgggcatttggtgagaatcaaagctgaccatggatccctggggccaacggcaacagcatgggcctcacctcctctgtgatttctttctttgcaccaaagacatcagtctccaacatgtttctgttttgttgtttgattcagcaaaaatctcccagtgacaacatcgcaatagttttttacttctcttaggtggctctgggatgggagaggggtaggatgtacaggggtagtttgttttagaaccagccgtattttacatgaagctgtataattaattgtcattatttttgttagcaaagattaaatgtgtcattggaagccatccctttttttacatttcatacaacagaaaccagaaaagcaatactgtttccattttaaggatatgattaatattattaatataataatgatgatgatgatgatgaaaactaaggatttttcaagagatctttctttccaaaacatttctggacagtacctgattgtattttttttttaaataaaagcacaagtacttttgaaaaaaaaccggaattc

SEQ ID NO: 12 - protein, 2201 aa, Homo sapiens
MGAMTQLLAGVFLAFLALATEGGVLKKVIRHKRQSGVNATLPEENQPVVFNHVYNIKLPVGSQCSVDLESASGEKDLAPPSEPSESFQEHTVDGENQIVFTHRINIPRRACGCAAAPDVKELLSRLEELENLVSSLREQCTAGAGCCLQPATGRLDTRPFCSGRGNFSTEGCGCVCEPGWKGPNCSEPECPGNCHLRGRCIDGQCICDDGFTGEDCSQLACPSDCNDQGKCVNGVCICFEGYAGADCSREICPVPCSEEHGTCVDGLCVCHDGFAGDDCNKPLCLNNCYNRGRCVENECVCDEGFTGEDCSELICPNDCFDRGRCINGTCYCEEGFTGEDCGKPTCPHACHTQGRCEEGQCVCDEGFAGLDCSEKRCPADCHNRGRCVDGRCECDDGFTGADCGELKCPNGCSGHGRCVNGQCVCDEGYTGEDCSQLRCPNDCHSRGRCVEGKCVCEQGFKGYDCSDMSCPNDCHQHGRCVNGMCVCDDGYTGEDCRDRQCPRDCSNRGLCVDGQCVCEDGFTGPDCAELSCPNDCHGQGRCVNGQCVCHEGFMGKDCKEQRCPSDCHGQGRCVDGQCICHEGFTGLDCGQHSCPSDCNNLGQCVSGRCICNEGYSGEDCSEVSPPKDLVVTEVTEETVNLAWDNEMRVTEYLVVYTPTHEGGLEMQFRVPGDQTSTIIQELEPGVEYFIRVFAILENKKSIPVSARVATYLPAPEGLKFKSIKETSVEVEWDPLDIAFETWEIIFRNMNKEDEGEITKSLRRPETSYRQTGLAPGQEYEISLHIVKNNTRGPGLKRVTTTRLDAPSQIEVKDVTDTTALITWFKPLAEIDGIELTYGIKDVPGDRTTIDLTEDENQYSIGNLKPDTEYEVSLISRRGDMSSNPAKETFTTGLDAPRNLRRVSQTDNSITLEWRNGKAAIDSYRIKYAPISGGDHAEVDVPKSQQATTKTTLTGLRPGTEYGIGVSAVKEDKESNPATINAATELDTPKDLQVSETAETSLTLLWKTPLAKFDRYRLNYSLPTGQWVGVQLPRNTTSYVLRGLEPGQEYNVLLTAEKGRHKSKPARVKASTEQAPELENLTVTEVGWDGLRLNWTAADQAYEHFIIQVQEANKVEAARNLTVPGSLRAVDIPGLKAATPYTVSIYGVIQGYRTPVLSAEASTGETPNLGEVVVAEVGWDALKLNWTAPEGAYEYFFIQVQEADTVEAAQNLTVPGGLRSTDLPGLKAATHYTITIRGVTQDFSTTPLSVEVLTEEVPDMGNLTVTEVSWDALRLNWTTPDGTYDQFTIQVQEADQVEEAHNLTVPGSLRSMEIPGLRAGTPYTVTLHGEVRGHSTRPLAVEVVTEDLPQLGDLAVSEVGWDGLRLNWTAADNAYEHFVIQVQEVNKVEAAQNLTLPGSLRAVDIPGLEAATPYRVSIYGVIRGYRTPVLSAEASTAKEPEIGNLNVSDITPESFNLSWMATDGIFETFTIEIIDSNRLLETVEYNISGAERTAHISGLPPSTDFIVYLSGLAPSIRTKTISATATTEALPLLENLTISDINPYGFTVSWMASENAFDSFLVTVVDSGKLLDPQEFTLSGTQRKLELRGLITGIGYEVMVSGFTQGHQTKPLRAEIVTEAEPEVDNLLVSDATPDGFRLSWTADEGVFDNFVLKIRDTKKQSEPLEITLLAPERTRDLTGLREATEYEIELYGISKGRRSQTVSAIATTAMGSPKEVIFSDITENSATVSWRAPTAQVESFRITYVPITGGTPSMVTVDGTKTQTRLVKLIPGVEYLVSIIAMKGFEESEPVSGSFTTALDGPSGLVTANITDSEALARWQPAIATVDSYVISYTGEKVPEITRTVSGNTVEYALTDLEPATEYTLRIFAEKGPQKSSTITAKFTTDLDSPRDLTATEVQSETALLTWRPPRASVTGYLLVYESVDGTVKEVIVGPDTTSYSLADLSPSTHYTAKIQALNGPLRSNMIQTIFTTIGLLYPFPKDCSQAMLNGDTTSGLYTIYLNGDKAQALEVFCDMTSDGGGWIVFLRRKNGRENFYQNWKAYAAGFGDRREEFWLGLDNLNKITAQGQYELRVDLRDHGETAFAVYDKFSVGDAKTRYKLKVEGYSGTAGDSMAYHNGRSFSTFDKDTDSAITNCALSYKGAFWYRNCHRVNLMGRYGDNNHSQGVNWFHWKGHEHSIQFAEMKLRPSNFRNLEGRRKRA

SEQ ID NO: 13 - DNA, 3672 bp, Homo sapiens
acatgtgcatatttcattccccaggcagacattttttagaaatcaatacatgccccaatattggaaagacttgttcttccacggtgactacagtacatgctgaagcgtgccgtttcagccctcatttaattcaatttgtaagtagcgcacgagcctctgtgggggaggataggctgaaaaaaaaaagtgggctcgtatttatctacaggactccatatagtcatatataggcatataaatctatgctttttctttgtttttttctttcttcctttctttcaaaggtttgcattaacttttcaaagtagttcctataggggcattgaggagcttcctcattctgggaaaactgagaaaacccatattctcctaatacaacccgtaatagcatttttgcctgcctcgaggcagagtttcccgtgagcaataaactcagcttttttgtggggcacagtactggatttgacagtgattccccacgtgtgttcatctgcacccaccgagccaggcagaggccagccctccgtggtgcacacagcacgcgcctcagtccatcccattttagtctttaaaccctcaggaagtcacagtctccggacaccacaccacattgagcccaacaggtccacgatggatccacctagtcccaccccagcctttttctttcatctgaacagaatgtgcatttttggaagcctccctcactctccatgctggcagagcaggagggagactgaagtaagagatggcagagggagatggtggcaaaaaggtttagatgcaggagaacagtaagatggatggttccggccagagtcgatgtggggaggaacagagggctgaagggagagggggctgactgttccattctagctttggcacaaagcagcagaaagggggaaaagccaatagaaatttccttagcttccccaccatatgtattttcatggatttgagaggaaagagaggaaaatgggggaatgggttgcaaaatagaaatgagcttaatccaggccgcagagccagggaaggtgagtaaccttaggagggtgctagactttagaagccagataggaagaatcagtctaaactggccatgctttggaagggacaagactatgtgctccgctgcccaccttcagcctgcaatgagggactgaggcccacgagtctttccagctcttcctccattctggccagtccctgcatcctccctggggtggaggatggaaggaaagctgggacaagcagggaacgcatgattcagggatgctgtcactcggcagccagattccgaaactcccattctccaatgacttcctcaaccaatgggtggccttgtgactgttctttaaggctgaagatatccaggaaagggggcttggacactggccaaggagaccccttcgtgctgtggacacagctctcttcactctttgctcatggcatgacacagcggagaccgcctccaacaacgaatttggggctacgaagaggaatagcgaaaaagcaaatctgtttcaactgatgggaaccctatagctatagaacttgggggctatctcctatgcccctggacaggacagttggctggggacaggagaagtgctcaatcttcatgagacaaaggggcccgatcaaggcagccacaaggccttgacctgccgagtcagcatgccccatctctctcgacagctgtcccctaaacccaactcacgtttctgtatgtcttaggccagtatcccaaacctcttccacgtcactgttctttccacccattctccctttgcatcttgagcagttatccaactaggatctgccaagtggatactggggtgccactcccctgagaaaagactgagccaggaactacaagctccccccacattcctcccagcctggacctaattcttgagaggggctctctcttcacggactgtgtctggactttgagcaggcttctgccccttgcgttggctctttgctgccagccatcaggtgggggattagagcctggtgtaagtgcgccagactcttccggtttccaaagttcgtgcctgcgaacccaaacctgtgagtctcttctgcatgcaggagtttctcctgggcagctggtcactccccagagaagctgggccttcatggacacatggaactaagcctcccaaatgggagttctggctgagcccagggtggggagatcctgggaagggaggcactggaggaagacggcacctcttcccccatggcagggtgtgagggaggcaggtttggaatggtgcgagtatggcaatctaagcaggggtctggtctctttgactccaggctcgctttggccgactgtctgctcacccagagaccttggactccggactatccatggctccgaatctaagtgctgcccactcccatgctcacacccacagaaggtcttcccatcccctttagattcgtgcctcactccaccagtgaggaagatgcctctgtctttcccacgactgccaggagatagggaagcccagccaggactgaccctccttcctccagcctgccctgacccacctggcaaagcagggcacatggggaggaagagactggaacctttctttgacagccaggcctagacagacaggcctggggacactggcccatgaggggaggaaggcaggcgcacgaggtccagggaggcccttttctgatcatgccccttctctcccaccccatctccccaccaccacctctgtggcctccatggtacccccacagggctggcctcccctagagggtgggcctcaaccacctcgtcccgccacgcaccggttagtgagacagggctgccacgcaaccgccaagcccccctcaaggtgggacagtaccccggacccatccactcactcctgagaggctccggcccagaatgggaacctcagagaagagctctaaggagaagaaaccccatagcgtcagagaggatatgtctggcttccaagagaaaggaggctccgttttgcaaagtggaggagggacgagggacaggggtttcaccagccagcaacctgggccttgtactgtctgtgtttttaaaaccactaaagtgcaagaattacattgcactgtttctccactttttattttctcttaggcttttgtttctatttcaaacatactttcttggttttctaatggagtatatagtttagtcatttcacagactctggcctcctctcctgaaatccttttggatggggaaagggaaggtggggagggtccgaggggaaggggaccccagcttccctgtgcccgctcaccccactccaccagtccccggtcgccagccggagtctcctctctaccgccactgtcacaccgtagcccacatggatagcacagttgtcagacaagattccttcagattccgagttgctaccggttgttttcgttgttgttgttgttgtttttctttttctttttttttttgaagacagcaataaccacagtacatattactgtagttctctatagttttacatacattcataccataactctgttctctcctcttttttgttttcaactttaaaaacaaaaataaacgatgataatctttactggtgaaaaggatggaaaaataaatcaacaaatgcaaccagtttgtgagaaaaaaaaaaaaaa

SEQ ID NO: 14 - protein, 272 aa, Homo sapiens
MVLLTAVLLLLAAYAGPAQSLGSFVHCEPCDEKALSMCPPSPLGCELVKEPGCGCCMTCALAEGQSCGVYTERCAQGLRCLPRQDEEKPLHALLHGRGVCLNEKSYREQVKIERDSREHEEPTTSEMAEETYSPKIFRPKHTRISELKAEAVKKDRRKKLTQSKFVGGAENTAHPRIISAPEMRQESEQGPCRRHMEASLQELKASPRMVPRAVYLPNCDRKGFYKRKQCKPSRGRKRGICWCVDKYGMKLPGMEYVDGDFQCHTFDSSNVE

SEQ ID NO: 15 - DNA, 1860 bp, Homo sapiens
gcggatcctcacacgactgtgatccgattctttccagcggcttctgcaaccaagcgggtcttacccccggtcctccgcgtctccagtcctcgcacctggaaccccaacgtccccgagagtccccgaatccccgctcccaggctacctaagaggatgagcggtgctccgacggccggggcagccctgatgctctgcgccgccaccgccgtgctactgagcgctcagggcggacccgtgcagtccaagtcgccgcgctttgcgtcctgggacgagatgaatgtcctggcgcacggactcctgcagctcggccaggggtgcgcgaacaccggagcgcacccgcagtcagctgagcgcgctggagcgcgcctgagcgcgtgcgggtccgcctgtcagggaaccgaggggtccaccgacctcccgttagcccctgagagccgggtggaccctgaggtccttcacagcctgcagacacaactcaaggctcagaacagcaggatccagcaactcttccacaaggtggcccagcagcagcggcacctggagaagcagcacctgcgaattcagcatctgcaaagccagtttggcctcctggaccacaagcacctagaccatgaggtggccaagcctgcccgaagaaagaggctgcccgagatggcccagccagttgacccggctcacaatgtcagccgcctgcaccggctgcccagggattgccaggagctgttccaggttggggagaggcagagtggactatttgaaatccagcctcaggggtctccgccatttttggtgaactgcaagatgacctcagatggaggctggacagtaattcagaggcgccacgatggctcagtggacttcaaccggccctgggaagcctacaaggcggggtttggggatccccacggcgagttctggctgggtctggagaaggtgcatagcatcacgggggaccgcaacagccgcctggccgtgcagctgcgggactgggatggcaacgccgagttgctgcagttctccgtgcacctgggtggcgaggacacggcctatagcctgcagctcactgcacccgtggccggccagctgggcgccaccaccgtcccacccagcggcctctccgtacccttctccacttgggaccaggatcacgacctccgcagggacaagaactgcgccaagagcctctctggaggctggtggtttggcacctgcagccattccaacctcaacggccagtacttccgctccatcccacagcagcggcagaagcttaagaagggaatcttctggaagacctggcggggccgctactacccgctgcaggccaccaccatgttgatccagcccatggcagcagaggcagcctcctagcgtcctggctgggcctggtcccaggcccacgaaagacggtgactcttggctctgcccgaggatgtggccaagaccacgactggagaagccccctttctgagtgcaggggggctgcatgcgttgcctcctgagatcgaggctgcaggatatgctcagactctagaggcgtggaccaaggggcatggagcttcactccttgctggccagggagttggggactcagagggaccacttggggccagccagactggcctcaatggcggactcagtcacattgactgacggggaccagggcttgtgtgggtcgagagcgccctcatggtgctggtgctgttgtgtgtaggtcccctggggacacaagcaggcgccaatggtatctgggcggagctcacagagttcttggaataaaagcaacctcagaacaaaaaaaaaaaaaaaaaagcggagctcacagagttcttggaataaaagcaacctcagaacaaaaaa

SEQ ID NO: 16 - protein, 405 aa, Homo sapiens [sequence omitted]

SEQ ID NO: 17 - DNA, 3901 bp, Homo sapiens
cagtttgcaaaagccagaggtgcaagaagcagcgactgcagcagcagcagcagcagcggcggtggcagcagcagcagcagcggcggcagcagcagcagcagcggaggcaccggtggcagcagcagcatcaccagcaacaacaacaaaaaaaaatcctcatcaaatcctcacctaagctttcagtgtatccagatccacatcttcactcaagccaggagagggaaagaggaaaggggggcaggaaaaaaaaaaaacccaacaacttagcggaaacttctcagagaatgctccaaaactcagcagtgcttctggtgctggtgatcagtgcttctgcaacccatgaggcggagcagaatgactctgtgagccccaggaaatcccgagtggcggctcaaaactcagctgaagtggttcgttgcctcaacagtgctctacaggtcggctgcggggcttttgcatgcctggaaaactccacctgtgacacagatgggatgtatgacatctgtaaatccttcttgtacagcgctgctaaatttgacactcagggaaaagcattcgtcaaagagagcttaaaatgcatcgccaacggggtcacctccaaggtcttcctcgccattcggaggtgctccactttccaaaggatgattgctgaggtgcaggaagagtgctacagcaagctgaatgtgtgcagcatcgccaagcggaaccctgaagccatcactgaggtcgtccagctgcccaatcacttctccaacagatactataacagacttgtccgaagcctgctggaatgtgatgaagacacagtcagcacaatcagagacagcctgatggagaaaattgggcctaacatggccagcctcttccacatcctgcagacagaccactgtgcccaaacacacccacgagctgacttcaacaggagacgcaccaatgagccgcagaagctgaaagtcctcctcaggaacctccgaggtgaggaggactctccctcccacatcaaacgcacatcccatgagagtgcataaccagggagaggttattcacaacctcaccaaactagtatcattttaggggtgttgacacaccaattttgagtgtactgtgcctggtttgatttttttaaagtagttcctattttctatcccccttaaagaaaattgcatgaaactaggcttctgtaatcaatatcccaacattctgcaatggcagcattcccaccaacaaaatccatgtgatcattctgcctctcctcaggagaaagtaccctcttttaccaacttcctctgccatgtcttttcccctgctcccctgagaccacccccaaacacaaaacattcatgtaactctccagccattgtaatttgaagatgtggatccctttagaacggttgccccagtagagttagctgataaggaaactttatttaaatgcatgtcttaaatgctcataaagatgttaaatggaattcgtgttatgaatctgtgctggccatggacgaatatgaatgtcacatttgaattcttgatctctaatgagctagtgtcttatggtcttgatcctccaatgtctaattttctttccgacacatttaccaaattgcttgagcctggctgtccaaccagactttgagcctgcatcttcttgcatctaatgaaaaacaaaaagctaacatctttacgtactgtaactgctcagagctttaaaagtatctttaacaattgtcttaaaaccagagaatcttaaggtctaactgtggaatataaatagctgaaaactaatgtactgtacataaattccagaggactctgcttaaacaaagcagtatataataactttattgcatatagatttagttttgtaacttagctttatttttcttttcctgggaatggaataactatctcacttccagatatccacataaatgctccttgtggccttttttataactaagggggtagaagtagttttaattcaacatcaaaacttaagatgggcctgtatgagacaggaaaaaccaacaggtttatctgaaggaccccaggtaagatgttaatctcccagcccacctcaacccagaggctactcttgacttagacctatactgaaagatctctgtcacatccaactggaaattccaggaaccaaaaagagcatccctatgggcttggaccacttacagtgtgataaggcctactatacattaggaagtggtagttctttactcgtcccctttcatcggtgcctggtactctggcaaatgatgatggggtgggagactttccattaaatcaatcaggaatgagtcaatcagcctttaggtctttagtccgggggacttggggctgagagagtataaataaccctgggctgtccagccttaatagacttctcttacattttcgtcctgtagcacgctgcctgccaaagtagtcctggcagctggaccatctctgtaggatcgtaaaaaaatagaaaaaaagaaaaaaaaaagaaagaaagagggaaaaagagctggtggtttgatcatttctgccatgatgtttacaagatggcgaccaccaaagtcaaacgactaacctatctatgaacaacagtagtttctcagggtcactgtccttgaacccaacagtcccttatgagcgtcactgcccaccaaaggtcaatgtcaagagaggaagagagggaggaggggtaggactgcaggggccactccaaactcgcttaggtagaaactattggtgctcgactctcactaggctaaactcaagatttgaccaaatcgagtgatagggatcctggtgggaggagagagggcacatctccagaaaaatgaaaagcaatacaactttaccataaagcctttaaaaccagtaacgtgctgctcaaggaccaagagcaattgcagcagacccagcagcagcagcagcagcacaaacattgctgcctttgtccccacacagcctctaagcgtgctgacatcagattgttaagggcatttttatactcagaactgtcccatccccaggtccccaaacttatggacactgccttagcctcttggaaatcaggtagaccatattctaagttagactcttcccctccctcccacacttcccacccccaggcaaggctgacttctctgaatcagaaaagctattaaagtttgtgtgttgtgtccattttgcaaacccaactaagccaggaccccaatgcgacaagtagttcatgagtattcctagcaaatttctctctttcttcagttcagtagatttccttttttcttttcttttttttttttttttttttttggctgtgacctcttcaaaccgtggtacccccccttttctccccacgatgatatctatatatgtatctacaatacatatatctacacatacagaaagaagcagttctcacatgttgctagttttttgcttctctttcccccaccctactccctccaattccccccttaaacttccaaagcttcgtcttgtgtttgctgcagagtgattcgggggctgacctagaccagtttgcatgattcttctcttgtgatttggttgcactttagacatttttgtgccattatatttgcattatgtatttataatttaaatgatatttaggtttttggctgagtactggaataaacagtgagcatatctggtatatgtcattatttattgttaaattacattttttaagctccatgtgcatataaaggttatgaaacatatcatggtaatgacagatgcaagttattttatttgcttattttttataattaaagatgccatagcataatatgaagcctttggtgaattccttctaagataaaaataataataaagtgttacgttttattggtttcaaaaaaaaaaaaaaaaaaaa

SEQ ID NO: 18 - protein, 247 aa, Homo sapiens
MLQNSAVLLVLVISASATHEAEQNDSVSPRKSRVAAQNSAEVVRCLNSALQVGCGAFACLENSTCDTDGMYDICKSFLYSAAKFDTQGKAFVKESLKCIANGVTSKVFLAIRRCSTFQRMIAEVQEECYSKLNVCSIAKRNPEAITEVVQLPNHFSNRYYNRLVRSLLECDEDTVSTIRDSLMEKIGPNMASLFHILQTDHCAQTHPRADFNRRRTNEPQKLKVLLRNLRGEEDSPSHIKRTSHESA

SEQ ID NO: 19 - DNA, 1993 bp, Homo sapiens
gtcagcagaagttacttcgagcacctatagtgatgaagacaggcctcccaaagtaccgccaagagaacctttgtcaccgagtaactcgcgcacaccgagtcccaaaagccttccgtcttacctcaatggggtcatgcccccgacacagagctttgcccctgatcccaagtatgtcagcagcaaagcactgcaaagacagaacagcgaaggatctgccagtaaggttccttgcattctgcccattattgaaaatgggaagaaggttagttcaacacattattacctactacctgaacgaccaccatacctggacaaatatgaaaaattttttagggaagcagaagaaacaaatggaggcgcccaaatccagccattacctgctgactgcggtatatcttcagccacagaaaagccagactcaaaaacaaaaatggatctgggtggccacgtgaagcgtaaacatttatcctatgtggtttctccttagaccttggggtcatggttcagcagaggttacataggagcaaatggttctcaattttccagtttgattgaagtgcagagaaaaatcccttagattgcaaaataaaatagttgaactctctgtcttcatgtggaaggtttagagcagttgtgagatgctgttatgctgagaaaccctgactttgttagtgttggaaaaaagtcttacaagtctataatttaaagatgtgatggtggggaggggaggatggggaagctttttatatatgcatacattacatacctatatataaacttgtggtataaccatagaccatagctgcaggttaaccaattagttactatcgtagagtaatatatattcagaataataaactcaagctggagaaatgagtcctgatagactgaaaattgagcaaatggaagaagatacagtattgtttagatcagaatcattaaaaaatatttttgtttagtaagtttgaagatttctggcttttaggccttttctattttgttccatttatttttgcaggcaatcttttccatggagggcagggtatccattctttaccatgggtgtacctgcttaggttaaaaatcataccaaggcctcatacttccaggtttcatgttgcgtcttgttgagggagggagagcaggttacttggcaaccatattgtcacctgtacctgtcacacatcttgaaaaataaaacgataatagaactagtgactaattttcccttacagttcctgcttggtcccacccactgaagtagctcatcgtagtgcgggccgtattagaggcagtggggtacgttagactcagatggaaaagtattctaggtgccagtgttaggatgtcagttttacaaaataatgaagcaattagctatgtgattgagagttattgtttggggatgtgtgttgtggttttgctttttttttttagactgtattaataaacatacaacacaagctggccttgtgttgctggttcctattcagtatttcctggggattgtttgctttttaagtaaaacacttctgacccatagctcagtatgtctgaattccagaggtcacatcagcatctttctgctttgaaaactctcacagctgtggctgcttcacttagatgcagtgagacacatagttggtgttccgattttcacatccttccatgtatttatcttgaagagataagcacagaagagaaggtgctcactaacagaggtacattactgcaatgttctcttaacagttaaacaagctgtttacagtttaaactgctgaatattatttgagctatttaaagcttattatattttagtatgaactaaatgaaggttaaaacatgcttaagaaaaatgcactgatttctgcattatgtgtacagtattggacaaaggattttattcattttgttgcattattttgaatattgtcttttcattttaataaagttataatacttatttatgataccattaaaaaaaaaaaaaaaa

SEQ ID NO: 20 - protein, 161 aa, Homo sapiens
SAEVTSSTYSDEDRPPKVPPREPLSPSNSRTPSPKSLPSYLNGVMPPTQSFAPDPKYVSSKALQRQNSEGSASKVPCILPIIENGKKVSSTHYYLLPERPPYLDKYEKFFREAEETNGGAQIQPLPADCGISSATEKPDSKTKMDLGGHVKRKHLSYVVSP

SEQ ID NO: 21 - DNA, 642 bp, Homo sapiens
tcgtgttcatgggagctcgttttcttttcctctaggcagagaagaggcgatggcggcgatggcatctctcggcgccctggcgctgctcctgctgtccagcctctcccgctgctcagccgaggcctgcctggagccccagatcaccccttcctactacaccacttctgacgctgtcatttccactgagaccgtcttcattgtggagatctccctgacatgcaagaacagggtccagaacatggctctctatgctgacgtcggtggaaaacaattccctgtcactcgaggccaggatgtggggcgttatcaggtgtcctggagcctggaccacaagagcgcccacgcaggcacctatgaggttagattcttcgacgaggagtcctacagcctcctcaggaaggctcagaggaataacgaggacatttccatcatcccgcctctgtttacagtcagcgtggaccatcggggcacttggaacgggccctgggtgtccactgaggtgctggctgcggcgatcggccttgtgatctactacttggccttcagtgcgaagagccacatccaggcctgagggcggcaccccagccctgcccttgcttccttcaataaacatcacaggacctgggactgcacaggaaaaaa

SEQ ID NO: 22 - protein, 173 aa, Homo sapiens
MAAMASLGALALLLLSSLSRCSAEACLEPQITPSYYTTSDAVISTETVFIVEISLTCKNRVQNMALYADVGGKQFPVTRGQDVGRYQVSWSLDHKSAHAGTYEVRFFDEESYSLLRKAQRNNEDISIIPPLFTVSVDHRGTWNGPWVSTEVLAAAIGLVIYYLAFSAKSHIQA

SEQ ID NO: 23 - DNA, 10 bp, Homo sapiens
tttgttaaaa

SEQ ID NO: 24 - DNA, 10 bp, Homo sapiens
gccacgttgt

SEQ ID NO: 25 - DNA, 10 bp, Homo sapiens
gtgctggtgc

SEQ ID NO: 26 - DNA, 10 bp, Homo sapiens
cagccaaata

SEQ ID NO: 27 - DNA, 10 bp, Homo sapiens
cttaagaaaa

SEQ ID NO: 28 - DNA, 10 bp, Homo sapiens
tgttagaaaa

SEQ ID NO: 29 - DNA, 10 bp, Homo sapiens
gatagcacag

SEQ ID NO: 30 - DNA, 10 bp, Homo sapiens
gctctctatg